(19) United States
(12) Patent Application Publication
(10) Pub. No.: US 2022/0265754 A1
(43) Pub. Date: Aug. 25, 2022

(54) METHOD OF IMPROVING DISEASES USING TRADITIONAL CHINESE MEDICINE POLYSACCHARIDES AND BACTERIAL COMPOSITION THEREOF

(71) Applicant: Multistars Biotechnology Company Limited, Taoyuan City (TW)

(72) Inventors: Po-I Wu, Taoyuan City (TW); Hsin-Chih Lai, Taoyuan City (TW); Chia-Chen Lu, Taoyuan City (TW); Tzu-Lung Lin, Taoyuan City (TW)

(21) Appl. No.: 17/675,169

(22) Filed: Feb. 18, 2022

Related U.S. Application Data
(60) Provisional application No. 63/151,123, filed on Feb. 19, 2021.

(51) Int. Cl. A61K 36/8984; A61K 36/074; A61K 36/062; A61K 35/741; A61P 3/04

(52) U.S. Cl. CPC A61K 36/8984; A61K 36/074; A61K 36/062; A61K 35/741; A61P 3/04; A61K 2236/331

(57) ABSTRACT

The present invention provides a method of improving chronic obstructive pulmonary disease and obesity using traditional Chinese medicine polysaccharides and bacterial composition thereof.

### CROSS-REFERENCE TO RELATED APPLICATION

This application claims priority of U.S. provisional application No. 63/151,123, filed on Feb. 19, 2021, the content of which are incorporated herein in its entirety by reference.

### BACKGROUND OF THE INVENTION

### 1. Field of the Invention

The present invention relates to methods of improving diseases using a traditional Chinese medicine polysaccharide and a bacterial composition thereof, and more particularly to methods of improving chronic obstructive pulmonary disease and obesity using Ganoderma lucidum (GL) polysaccharide, Dendrobium huoshanense (DH) polysaccharide, Hirsutella sinensis (HSM) polysaccharide, or any combination thereof.

### 2. The Prior Art

Due to the limited number of digestive enzymes encoded by the human genome, polysaccharides in ingested foods are often not fully digested in the small intestine before reaching the rectum. Microbes in the gut can decompose the polysaccharides through glycolysis, and produce many short-chain oligosaccharides with different lengths, structures, and branching numbers, some of which can even act as prebiotics to promote gut microbes growth. The shorter polysaccharides can be further digested into monosaccharides such as glucose, arabinose, or rhamnose, which can also be used to promote the growth of intestinal microorganisms. The monosaccharides can also be further metabolized into short-chain fatty acids, lactic acid, hydrogen and other metabolites in the intestine, which may directly affect the physiological functions of the hosts.

However, since different types of bacteria have different carbohydrate-active enzymes (CAZymes), the extent to which polysaccharides in food are digested and utilized by individual gut bacteria, and the impact of the resulting metabolites on host physiology, and even on the effects of different diseases needs further research.

### SUMMARY OF THE INVENTION

One objective of the present invention is to provide a method of ameliorating chronic obstructive pulmonary disease (COPD), comprising administering to a subject in need thereof a composition containing a traditional Chinese medicine polysaccharide, wherein the traditional Chinese medicine polysaccharide is selected from a group consisting of Ganoderma lucidum polysaccharide, Dendrobium huoshanense polysaccharide, Hirsutella sinensis polysaccharide, and any combination thereof.

In one embodiment of the present invention, the Ganoderma lucidum polysaccharide is from a water extract of Ganoderma lucidum, the Dendrobium huoshanense polysaccharide is from a water extract of Dendrobium huoshanense, and the Hirsutella sinensis polysaccharide is from a water extract of Hirsutella sinensis.

In the embodiment of the present invention, an effective amount of the traditional Chinese medicine polysaccharide is at least twice a week for the subject in need thereof with at least 25 mg/kg each time.

The further objective of the present invention is to provide a method of ameliorating chronic obstructive pulmonary disease, comprising administering to a subject in need thereof a bacterial composition containing a bacterial consortium and the traditional Chinese medicine polysaccharide above.

In the embodiment of the present invention, the composition or the bacterial composition is ameliorating body weight loss, abnormal lung histopathology, and/or abnormal lung function of the subject caused by chronic obstructive pulmonary disease.

The other objective of the present invention is to provide a method of preventing and/or treating obesity, comprising administering to a subject in need thereof a bacterial composition containing a bacterial consortium and the traditional Chinese medicine polysaccharide above.

In the embodiments of the present invention, the bacterial consortium is consisted of a Bacteroidetes bacterium; moreover, the Bacteroidetes bacterium is selected from a group consisting of Parabacteroides goldsteinii (P. goldsteinii), Parabacteroides distasonis (P. distasonis), Bacteroides xylanisolvens (B. xylanisolvens), Bacteroides uniformis (B. uniformis), Bacteroides fragilis (B. fragilis), Bacteroides ovatus (B. ovatus), and any combination thereof.

In other embodiment of the present invention, the bacterial composition reduces body weight gain of the subject in need thereof; and the bacterial composition reduces adipose tissue weight, triglyceride, total cholesterol, and/or fasting glucose of the subject in need thereof.

In the embodiments of the present invention, an effective amount of the bacterial composition is at least twice a week for the subject in need thereof with the bacterial composition containing at least 3×10¹⁰ CFU/kg of the bacterial consortium and at least 25 mg/kg of the bacterial composition each time.

In the embodiments of the present invention, the composition or the bacterial composition further comprises a pharmaceutically acceptable excipient, carrier, adjuvant, or food additive.

In the embodiments of the present invention, the composition or the bacterial composition is in the form of a spray, a solution, a semi-solid preparation, a solid preparation, a gelatin capsule, a soft capsule, a tablet, a chewing gum, or a freeze-dried powder preparation.

In the present invention, the bacterial consortium consisted of Bacteroidetes can effectively utilize and digest the traditional Chinese medicine polysaccharide of the present invention, and the traditional Chinese medicine polysaccharide of the present invention and the bacterial composition of the present invention containing the traditional Chinese medicine polysaccharide can effectively inhibit inflammatory responses, improve chronic obstructive pulmonary disease, and prevent and/or treat diet-induced obesity and metabolic syndrome. In addition, previous studies have shown that synbiotic which was prepared by mixing polysaccharides and bacteria could not maintain the effects of the polysaccharides or the bacteria themselves. However, the results of the present invention show that the bacterial composition of the present invention which was prepared by mixing the traditional Chinese medicine polysaccharides and the bacterial consortium of the present invention would provide the significantly better effects.

The embodiments of the present invention are further described with the following drawings. The following embodiments are given to illustrate the present invention and are not intended to limit the scope of the present invention, and one with ordinary skill in the art can make some modifications and refinements without departing from the spirit and scope of the present invention. Therefore, the scope of the present invention is defined by the scope of the appended claims.

### BRIEF DESCRIPTION OF THE DRAWINGS

FIG. 1 shows the growth curve of the bacterial consortium of the present invention over time in the minimal medium supplemented with the Ganoderma lucidum polysaccharide of the present invention. GLPS represented the Ganoderma lucidum polysaccharide of the present invention.

FIG. 2 shows the growth curve of the bacterial consortium of the present invention over time in the minimal medium supplemented with the Dendrobium huoshanense polysaccharide of the present invention. DHPS represented the Dendrobium huoshanense polysaccharide of the present invention.

FIG. 3 shows the growth curve of the bacterial consortium of the present invention over time in the minimal medium supplemented with the Hirsutella sinensis polysaccharide of the present invention. HSMPS represented the Hirsutella sinensis polysaccharide of the present invention.

FIG. 4 shows the effects of the Ganoderma lucidum polysaccharide and the bacterial composition containing the Ganoderma lucidum polysaccharide of the present invention on promoting the secretion of IL-10 from macrophages. CTL represented the control group only treated with PBS; GLPS represented the experimental group treated with the Ganoderma lucidum polysaccharide of the present invention; Bacteroidetes bacteria represented the experimental group treated with the bacterial consortium of the present invention; Bacteroidetes bacteria+GLPS represented the experimental group treated with the bacterial composition of the present invention containing the Ganoderma lucidum polysaccharide.

FIG. 5 shows the effects of the Dendrobium huoshanense polysaccharide and the bacterial composition containing the Dendrobium huoshanense polysaccharide of the present invention on promoting the secretion of IL-10 from macrophages. CTL represented the control group only treated with PBS; DHPS represented the experimental group treated with the Dendrobium huoshanense polysaccharide of the present invention; Bacteroidetes bacteria represented the experimental group treated with the bacterial consortium of the present invention; Bacteroidetes bacteria+DHPS represented the experimental group treated with the bacterial composition of the present invention containing the Dendrobium huoshanense polysaccharide.

FIG. 6 shows the effects of the Hirsutella sinensis polysaccharide and the bacterial composition containing the Hirsutella sinensis polysaccharide of the present invention on promoting the secretion of IL-10 from macrophages. CTL represented the control group only treated with PBS; HSMPS represented the experimental group treated with the Hirsutella sinensis polysaccharide of the present invention; Bacteroidetes bacteria represented the experimental group treated with the bacterial consortium of the present invention; Bacteroidetes bacteria+HSMPS represented the experimental group treated with the bacterial composition of the present invention containing the Hirsutella sinensis polysaccharide.

FIGS. 7A and 7B show the effects of the Ganoderma lucidum polysaccharide and the bacterial composition containing the Ganoderma lucidum polysaccharide of the present invention on improving the body weight loss in subjects with COPD.

FIG. 8A is the histological image showing the effects of the Ganoderma lucidum polysaccharide and the bacterial composition containing the Ganoderma lucidum polysaccharide of the present invention on improving the abnormal lung histopathology in subjects with COPD.

FIG. 8B is the analysis results according to the histological image of FIG. 8A.

FIG. 9A shows the effects of the Ganoderma lucidum polysaccharide and the bacterial composition containing the Ganoderma lucidum polysaccharide of the present invention on improving abnormality of FRC in the subjects with COPD.

FIG. 9B shows the effects of the Ganoderma lucidum polysaccharide and the bacterial composition containing the Ganoderma lucidum polysaccharide of the present invention on improving abnormality of FVC in the subjects with COPD.

FIG. 9C shows the effects of the Ganoderma lucidum polysaccharide and the bacterial composition containing the Ganoderma lucidum polysaccharide of the present invention on improving abnormality of Cchord in the subjects with COPD.

FIG. 9D shows the effects of the Ganoderma lucidum polysaccharide and the bacterial composition containing the Ganoderma lucidum polysaccharide of the present invention on improving abnormality of FEV100/FVC ration in the subjects with COPD.

FIGS. 10A and 10B show the effects of the Dendrobium huoshanense polysaccharide and the bacterial composition containing the Dendrobium huoshanense polysaccharide of the present invention on improving the body weight loss in subjects with COPD.

FIG. 11A is the histological image showing the effects of the Dendrobium huoshanense polysaccharide and the bacterial composition containing the Dendrobium huoshanense polysaccharide of the present invention on improving the abnormal lung histopathology in subjects with COPD.

FIG. 11B is the analysis results according to the histological image of FIG. 11A.

FIG. 12A shows the effects of the Dendrobium huoshanense polysaccharide and the bacterial composition containing the Dendrobium huoshanense polysaccharide of the present invention on improving abnormality of FRC in the subjects with COPD.

FIG. 12B shows the effects of the Dendrobium huoshanense polysaccharide and the bacterial composition containing the Dendrobium huoshanense polysaccharide of the present invention on improving abnormality of FVC in the subjects with COPD.

FIG. 12C shows the effects of the Dendrobium huoshanense polysaccharide and the bacterial composition containing the Dendrobium huoshanense polysaccharide of the present invention on improving abnormality of Cchord in the subjects with COPD.

FIG. 12D shows the effects of the Dendrobium huoshanense polysaccharide and the bacterial composition containing the Dendrobium huoshanense polysaccharide of the present invention on improving abnormality of FEV100/FVC ration in the subjects with COPD.

FIGS. 13A and 13B show the effects of the Hirsutella sinensis polysaccharide and the bacterial composition containing the Hirsutella sinensis polysaccharide of the present invention on improving the body weight loss in subjects with COPD.

FIG. 14A is the histological image showing the effects of the Hirsutella sinensis polysaccharide and the bacterial composition containing the Hirsutella sinensis polysaccharide of the present invention on improving the abnormal lung histopathology in subjects with COPD.

FIG. 14B is the analysis results according to the histological image of FIG. 14A.

FIG. 15A shows the effects of the Hirsutella sinensis polysaccharide and the bacterial composition containing the Hirsutella sinensis polysaccharide of the present invention on improving abnormality of FRC in the subjects with COPD.

FIG. 15B shows the effects of the Hirsutella sinensis polysaccharide and the bacterial composition containing the Hirsutella sinensis polysaccharide of the present invention on improving abnormality of FVC in the subjects with COPD.

FIG. 15C shows the effects of the Hirsutella sinensis polysaccharide and the bacterial composition containing the Hirsutella sinensis polysaccharide of the present invention on improving abnormality of Cchord in the subjects with COPD.

FIG. 15D shows the effects of the Hirsutella sinensis polysaccharide and the bacterial composition containing the Hirsutella sinensis polysaccharide of the present invention on improving abnormality of FEV100/FVC ration in the subjects with COPD.

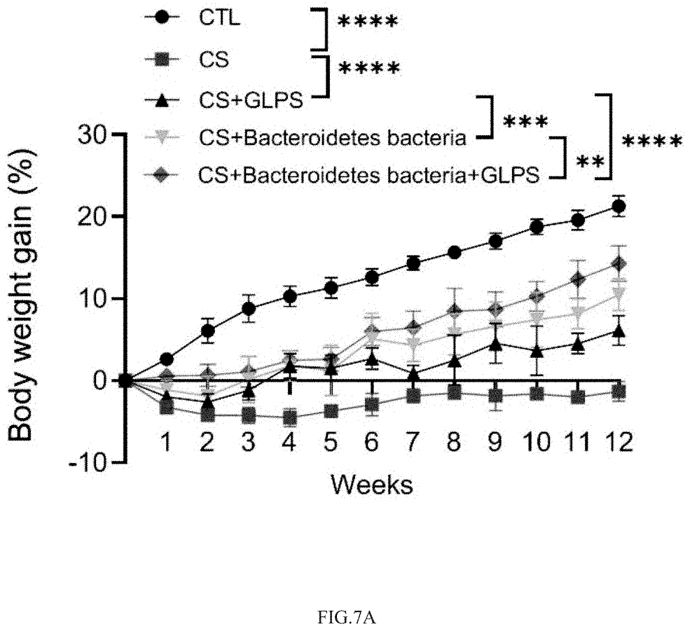

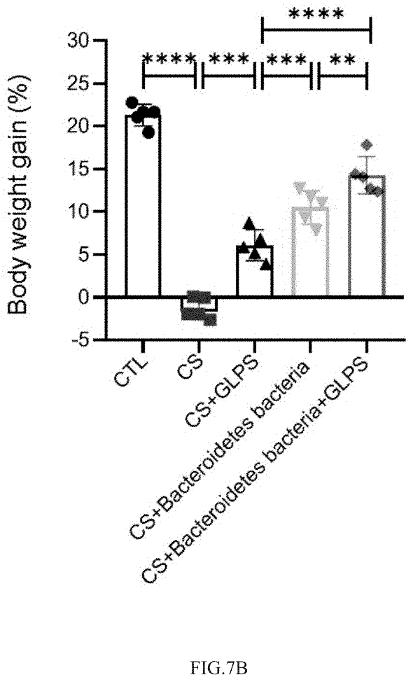

In FIGS. 7A to 15D above, CTL represents mice of the control group which were not treated with cigarette smoke nor with the traditional Chinese medicine polysaccharide, the bacterial consortium, or the bacterial composition; CS represents mice of the comparative group which were treated with cigarette smoke but not with the traditional Chinese medicine polysaccharide, the bacterial consortium, or the bacterial composition; CS+Bacteroidetes bacteria represents mice of the experimental group which were treated with cigarette smoke and with the bacterial consortium of the present invention; CS+GLPS represents mice of the experimental group which were treated with cigarette smoke and with the Ganoderma lucidum polysaccharide of the present invention; CS+Bacteroidetes bacteria+GLPS represents mice of the experimental group which were treated with cigarette smoke and with the bacterial composition of the present invention containing the Ganoderma lucidum polysaccharide; CS+DHPS represents mice of the experimental group which were treated with cigarette smoke and with the Dendrobium huoshanense polysaccharide of the present invention; CS+Bacteroidetes bacteria+DHPS represents mice of the experimental group which were treated with cigarette smoke and with the bacterial composition of the present invention containing the Dendrobium huoshanense polysaccharide; CS+HSMPS represents mice of the experimental group which were treated with cigarette smoke and with the Hirsutella sinensis polysaccharide of the present invention; CS+Bacteroidetes bacteria+HSMPS represents mice of the experimental group which were treated with cigarette smoke and with the bacterial composition of the present invention containing the Hirsutella sinensis polysaccharide.

FIGS. 16A and 16B show the effects of the Ganoderma lucidum polysaccharide and the bacterial composition containing the Ganoderma lucidum polysaccharide of the present invention on reducing body weight gain in obese subjects.

FIG. 17 shows the effects of the Ganoderma lucidum polysaccharide and the bacterial composition containing the Ganoderma lucidum polysaccharide of the present invention on reducing adipose tissue weight in obese subjects.

FIG. 18A shows the effects of the Ganoderma lucidum polysaccharide and the bacterial composition containing the Ganoderma lucidum polysaccharide of the present invention on reducing the triglyceride level in blood of obese subjects.

FIG. 18B shows the effects of the Ganoderma lucidum polysaccharide and the bacterial composition containing the Ganoderma lucidum polysaccharide of the present invention on reducing the total cholesterol level in blood of obese subjects.

FIG. 18C shows the effects of the Ganoderma lucidum polysaccharide and the bacterial composition containing the Ganoderma lucidum polysaccharide of the present invention on reducing the fasting glucose level in blood of obese subjects.

FIGS. 19A and 19B show the effects of the Dendrobium huoshanense polysaccharide and the bacterial composition containing the Dendrobium huoshanense polysaccharide of the present invention on reducing body weight gain in obese subjects.

FIG. 20 shows the effects of the Dendrobium huoshanense polysaccharide and the bacterial composition containing the Dendrobium huoshanense polysaccharide of the present invention on reducing adipose tissue weight in obese subjects.

FIG. 21A shows the effects of the Dendrobium huoshanense polysaccharide and the bacterial composition containing the Dendrobium huoshanense polysaccharide of the present invention on reducing the triglyceride level in blood of obese subjects.

FIG. 21B shows the effects of the Dendrobium huoshanense polysaccharide and the bacterial composition containing the Dendrobium huoshanense polysaccharide of the present invention on reducing the total cholesterol level in blood of obese subjects.

FIG. 21C shows the effects of the Dendrobium huoshanense polysaccharide and the bacterial composition containing the Dendrobium huoshanense polysaccharide of the present invention on reducing the fasting glucose level in blood of obese subjects.

FIGS. 22A and 22B show the effects of the Hirsutella sinensis polysaccharide and the bacterial composition containing the Hirsutella sinensis polysaccharide of the present invention on reducing body weight gain in obese subjects.

FIG. 23 shows the effects of the Hirsutella sinensis polysaccharide and the bacterial composition containing the Hirsutella sinensis polysaccharide of the present invention on reducing adipose tissue weight in obese subjects.

FIG. 24A shows the effects of the Hirsutella sinensis polysaccharide and the bacterial composition containing the Hirsutella sinensis polysaccharide of the present invention on reducing the triglyceride level in blood of obese subjects.

FIG. 24B shows the effects of the Hirsutella sinensis polysaccharide and the bacterial composition containing the Hirsutella sinensis polysaccharide of the present invention on reducing the total cholesterol level in blood of obese subjects.

FIG. 24C shows the effects of the Hirsutella sinensis polysaccharide and the bacterial composition containing the Hirsutella sinensis polysaccharide of the present invention on reducing the fasting glucose level in blood of obese subjects.

In FIGS. 16A to 24C above, Chow represents mice of the control group which were fed with standard chow diet and were not treated with the traditional Chinese medicine polysaccharide, the bacterial consortium, or the bacterial composition; HFD represents mice of the comparative group which were fed with high fat diet (HFD) but were not treated with the traditional Chinese medicine polysaccharide, the bacterial consortium, or the bacterial composition; HFD+Bacteroidetes bacteria represents mice of the experimental group which were fed with high fat diet (HFD) and were treated with the bacterial consortium of the present invention; HFD+GLPS represents mice of the experimental group which were fed with high fat diet (HFD) and were treated with the Ganoderma lucidum polysaccharide of the present invention; HFD+Bacteroidetes bacteria+GLPS represents mice of the experimental group which were fed with high fat diet (HFD) and were treated with the bacterial composition of the present invention containing the Ganoderma lucidum polysaccharide; HFD+DHPS represents mice of the experimental group which were fed with high fat diet (HFD) and were treated with the Dendrobium huoshanense polysaccharide of the present invention; HFD+Bacteroidetes bacteria+DHPS represents mice of the experimental group which were fed with high fat diet (HFD) and were treated with the bacterial composition of the present invention containing the Dendrobium huoshanense polysaccharide; HFD+HSMPS represents mice of the experimental group which were fed with high fat diet (HFD) and were treated with the Hirsutella sinensis polysaccharide of the present invention; HFD+Bacteroidetes bacteria+HSMPS represents mice of the experimental group which were fed with high fat diet (HFD) and were treated with the bacterial composition of the present invention containing the Hirsutella sinensis polysaccharide.

### DETAILED DESCRIPTION OF THE PREFERRED EMBODIMENT

All technical and scientific terms used herein, unless otherwise defined, have the meaning commonly understood by one with ordinary skill in the art.

Statistical analysis was performed using Excel software. Data were expressed as mean±standard deviation (SD) or mean±interquartile range (IQR), and the Newman-Keuls multiple comparison post hoc one-way ANOVA analysis or the Turkey's multiple comparison post hoc one-way ANOVA analysis was used to analyze whether the sample mean between each group was statistically significant.

The data provides in the present invention represent approximated, experimental values that vary within a range of ±20%, preferably ±10%, and most preferably ±5%.

As used herein, the term “bacterial consortium” refers to a mixture of a plurality of bacteria; and the term “bacterial consortium” refers to a mixture of the bacterial consortium and a traditional Chinese medicine polysaccharide.

The traditional Chinese medicine polysaccharide of the present invention or the bacterial composition of the present invention containing the traditional Chinese medicine polysaccharide can be applied to a preparation of a pharmaceutical composition for preventing and/or treating chronic obstructive pulmonary disease, or preventing and/or treating obesity; wherein, the pharmaceutical composition may be a medicine, a nutritional supplement, a health food, or any combination thereof, and may further include a pharmaceutically acceptable excipient, carrier, adjuvant, and/or food additives.

In one preferred embodiment of the present invention, the traditional Chinese medicine polysaccharide or the bacterial composition containing the traditional Chinese medicine polysaccharide is formulated in a pharmaceutically acceptable vehicle, and is made into a suitable dosage form of an oral administration, and the pharmaceutical composition is preferably in a dosage form selected from the following group: a solution, a suspension, a powder, a tablet, a pill, a syrup, a lozenge, a troche, a chewing gum, a capsule, and the like.

According to the present invention, the pharmaceutically acceptable vehicle may include one or more reagents selected from the following: a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome, and the like. The selection and quantity of these reagents is a matter of professionalism and routine for one with ordinary skill in the art.

According to the present invention, the pharmaceutically acceptable vehicle may include a solvent selected from the group consisting of: water, normal saline, phosphate buffered saline (PBS), aqueous solution containing alcohol, and combinations thereof.

In another preferred embodiment of the present invention, the traditional Chinese medicine polysaccharide or the bacterial composition containing the traditional Chinese medicine polysaccharide can be prepared into a food product, and be formulated with edible materials which include but not limited to: beverages, fermented foods, bakery products, health foods, nutritional supplements, and dietary supplements.

According to the present invention, the operating procedures and parameter conditions for bacterial culture are within the professional literacy and routine techniques of one with ordinary skill in the art.

According to the present invention, the operating procedures and parameter conditions for intraperitoneal injection in animals are within the professional literacy and routine techniques of one with ordinary skill in the art.

According to the present invention, the operating procedures and parameter conditions for Buxco research systems in animals are within the professional literacy and routine techniques of one with ordinary skill in the art.

Material and Method

### 1. Preparation of the Traditional Chinese Medicine (TCM) Polysaccharide

In the embodiments of the present invention, the traditional Chinese medicine (TCM) used herein may be but not limited to: Ganoderma lucidum (GL), Dendrobium huoshanense (DH), Hirsutella sinensis (HSM), or any composition thereof.

1.1 Preparation of the Ganoderma lucidum Polysaccharides (GLPS)
1.1.1 Preparation of the Concentrated Water Extract of Ganoderma lucidum

First, a mixture (50 g/L) was prepared by mixing 500 g of dried Ganoderma lucidum mycelium into 10 liters of distilled water using a 20 liter-stirred tank reactor. The 5.0% (w/v) mixture was agitated at a speed of 150 rpm for 30 mm at 121° C. The mixture was then centrifuged to remove insoluble material and to obtain a Ganoderma lucidum water extract. The Ganoderma lucidum water extract was concentrated to a final volume of 2.5 L using a vacuum concentrator. The concentrated Ganoderma lucidum water extract was sterilized at high temperature and pressure for 20 minutes in an autoclave to obtain a 20% (w/v) concentrated Ganoderma lucidum water extract and stored at 4° C.

1.1.2 Preparation of the Ganoderma lucidum Crude Polysaccharide Extract

Next, 120 mL of the 20% (w/v) concentrated Ganoderma lucidum water extract was mixed with 5 volumes (600 mL) of 95% ethanol and incubated at 4° C. for 16 hours to induce the precipitation of crude polysaccharide. The mixture was centrifuged to obtain a supernatant and a precipitate (pellet). The supernatant was removed, while 120 mL of 70% ice-cold ethanol was used to wash and re-suspend the precipitate to obtain a mixture. Repeat such washing-resuspension-centrifugation steps three times and the mixtures were combined. The crude polysaccharide precipitate (pellet) was dissolved into 1,000 mL of distilled water and concentrated to a final volume of 700 mL using a vacuum concentrator in order to remove residual ethanol. Finally, distilled water was added to obtain a Ganoderma lucidum crude polysaccharide extract with a final volume of 2,400 mL.

1.1.3 Fractionation of the Ganoderma lucidum Crude Polysaccharide Extract

2,400 mL of the Ganoderma lucidum crude polysaccharide extract was placed into a beaker, followed by incubation at 50° C. into a water bath. The Ganoderma lucidum crude polysaccharide extract was fractionated by using a tangential flow filtration (TFF) system (KrosFlo, Spectrum Laboratories) with a 0.2-nm hollow fiber membrane (1,500 cm², polyethersulfone, PES). The trans-membrane pressure (TMP) was set at 15-16 psi. 600 mL of distilled water was added into the retentate during filtration when the retentate remained from 800 to 1000 mL for continuing the filtration. Addition of water was repeated two times (a total of 1,800 mL distilled water was added to the retentate). 650 mL of sub-fraction G1-1 and 3,600 mL of filtrate were obtained this way.

The above-mentioned 3,600 mL of 0.2-μm filtrate was placed into a beaker and incubated at 50° C. in a water bath. The 3,600 mL of filtrate was fractionated by using the TFF system with a 300-kDa cassette membrane (50 cm², PES). The TMP was set at 16-18 psi. 600 mL of distilled water was added into the retentate during filtration when the retentate remained from 1,000 to 1,200 mL for continuing the filtration. 950 mL of sub-fraction G1-2 and 3,600 mL of filtrate were obtained. Sub-fractions G1-1 and G1-2 were combined to obtain 1,600 mL of final sub-fraction G1 which was the Ganoderma lucidum Polysaccharides (GLPS) of the present invention.

1.2 Preparation of the Dendrobium huoshanense Polysaccharides (DHPS)

First, the freeze-dried 2000 g of Dendrobium huoshanense were pulverized and defatted twice with petroleum ether in Soxhlet extractor. The dregs were successively extracted thrice with boiling distilled-water, with each time for 0.5 hour. The Dendrobium huoshanense water extracts were combined and followed by centrifuging at 8000 rpm for 10 minutes at 4° C. The supernatant was subsequently concentrated in a rotary evaporator at 60° C. to small volumes under reduced pressure, and precipitated with four volumes of anhydrous ethanol for overnight. The precipitate was collected by centrifugation and washed three times with acetone. Afterwards, the precipitate was re-suspended in distilled-water and de-proteinized using the Sevag reagent. The aqueous fraction was precipitated with anhydrous ethanol at the ratio of 1:4 (v:v) for 24 hours at 4° C. The resulting precipitate was harvested by centrifugation, dialysis and lyophilization to produce polysaccharides which was the Dendrobium huoshanense Polysaccharides (DHPS) of the present invention.

1.3 Preparation of the Hirsutella sinensis Polysaccharides (HSMPS)
1.3.1 Preparation of the Concentrated Water Extract of Hirsutella sinensis

Hirsutella sinensis is the anamorphic mycelium (or Hirsutella sinensis mycelium) form of Ophiocordyceps sinensis fruiting bodies. First, a mixture (50 g/L) was prepared by mixing 500 g of dried Hirsutella sinensis mycelium into 10 liters of distilled water using a 20 liter-stirred tank reactor. The 5% (w/v) mixture was agitated at a speed of 150 rpm for 30 minutes at 121° C. The mixture was then centrifuged to remove insoluble material and to obtain a Hirsutella sinensis water extract. The Hirsutella sinensis water extract was concentrated to a final volume of 2.5 L using a vacuum concentrator. The concentrated Hirsutella sinensis water extract was sterilized at high temperature and pressure for 20 minutes in an autoclave to obtain a 20% (w/v) concentrated Hirsutella sinensis water extract water extract and stored at 4° C.

1.3.2 Preparation of the Hirsutella sinensis Crude Polysaccharide Extract

Next, 120 mL of the 20% (w/v) concentrated Hirsutella sinensis water extract was mixed with 5 volumes (600 mL) of 95% ethanol and incubated at 4° C. for 16 hours to induce the precipitation of crude polysaccharide. The mixture was centrifuged to obtain a supernatant and a precipitate (pellet). The supernatant was removed, while 120 mL of 70% ice-cold ethanol was used to wash and re-suspend the precipitate to obtain a mixture. Repeat such washing-resuspension-centrifugation steps three times and the mixtures were combined. The crude polysaccharide precipitate (pellet) was dissolved into 1,000 mL of distilled water and concentrated to a final volume of 700 mL using a vacuum concentrator in order to remove residual ethanol. Finally, distilled water was added to obtain a Hirsutella sinensis crude polysaccharide extract with a final volume of 2,400 mL.

1.3.3 Fractionation of the Hirsutella sinensis Crude Polysaccharide Extract

Next, 2,400 mL of the Hirsutella sinensis crude polysaccharide extract was placed into a beaker, followed by incubation at 50° C. into a water bath. The Hirsutella sinensis crude polysaccharide extract was fractionated by using a tangential flow filtration (TFF) system (KrosFlo, Spectrum Laboratories) with a 0.2-μm hollow fiber membrane (1,500 cm², PES). The trans-membrane pressure (TMP) was set at 15-16 psi. 600 mL of distilled water was added into the retentate during filtration when the retentate remained from 800 to 1000 mL for continuing the filtration. Addition of water was repeated two times (a total of 1,800 mL distilled water was added to the retentate). 1250 mL of sub-fraction GS-1-1 and 3,600 mL of filtrate were obtained this way.

The above-mentioned 3,600 mL of 0.2-μm filtrate was placed into a beaker and incubated at 50° C. in a water bath. The 3,600 mL of filtrate was fractionated by using the TFF system with a 300-kDa cassette membrane (50 cm², PES). The TMP was set at 18-20 psi. 600 mL of distilled water was added into the retentate during filtration when the retentate remained from 1,000 to 1,200 mL for continuing the filtration. 1,040 mL of sub-fraction GS-1-2 and 3,600 mL of filtrate were obtained. Sub-fractions GS-1-1 and GS-1-2 were combined to obtain 2,290 mL of final sub-fraction GS-1 which was the Hirsutella sinensis Polysaccharides (HSMPS) of the present invention.

### 2. Bacterial Cultivation of Bacteroidetes

Bacteroidetes are anaerobic bacteria and need to be cultured in an anaerobic incubator at 37° C. In the embodiments of the present invention, a Whitley DG250 anaerobic chamber (Don Whitley, Bingley, UK) was used to cultivate bacteria of Bacteroidetes, wherein the anaerobic chamber contained 5% carbon dioxide, 5% hydrogen, and 90% nitrogen, and an anaerobic indicator (Oxoid, Hampshire, UK) was used to confirm anaerobic conditions. The liquid culture medium for the bacteria was thioglycollate medium (BD, USA, #225710), and the solid medium was anaerobic blood agar (Ana. BAP) (Creative, New Taipei city, Taiwan). The bacteria could be stored in a refrigerator at −80° C. for a long-term preservation, and the protective liquid was 25% glycerin. There was no need for special cooling treatment, and could be stored by freeze-drying to stabilize their activities.

### 3. Preparation of the Bacterial Consortium

The bacterial consortium used in the embodiments of the present invention may be composed of bacteria form the phylum Bacteroidetes; and more particularly, the bacterial consortium of the present invention may be composed of Parabacteroides goldsteinii (P. goldsteinii), Parabacteroides distasonis (P. distasonis), Bacteroides xylanisolvens (B. xylanisolvens), Bacteroides uniformis (B. uniformis), Bacteroides fragilis (B. fragilis), and/or Bacteroides ovatus (B. ovatus).

In preferred embodiments of the present invention, P. goldsteinii may be the DSM 32939 strain (patent deposit has been completed in US20200078414A1, referred to herein as MTS01 strain), P. distasonis may be the ATCC8503 strain (purchased from American Type Culture Collection, ATCC), B. xylanisolvens may be the DSM18836 strain (purchased from Deutsche Sammlung von Mikroorganismen and Zellkulturen, DSMZ), B. uniformis may be the ATCC8492 strain (purchased from ATCC), B. fragilis may be the NCTC9343 strain (purchased from the United Kingdom National Culture Collection, NCTC), and B. ovatus may be the ATCC8483 strain (purchased from ATCC).

In the embodiments of the present invention, the aforementioned bacteria constituting the bacterial consortium of the present invention may be co-cultured by the aforementioned bacterial cultivation method of Bacteroidetes, and then be prepared into any dosage form to be the bacterial consortium of the present invention; alternatively, the each bacterium may be cultured separately and mixed together, and then be prepared into any dosage form to be the bacterial consortium of the present invention.

### 4. Preparation of the Bacterial Composition

The bacterial composition used in the embodiments of the present invention may be composed of a traditional Chinese medicine polysaccharide and a bacterial consortium. The traditional Chinese medicine polysaccharide may be the Ganoderma lucidum polysaccharide, the Dendrobium huoshanense polysaccharide, the Hirsutella sinensis polysaccharide, or any combination thereof, and the bacterial consortium may be the aforementioned bacteria from Bacteroidetes.

The following would be described in detail: the test of the utilization and digestion of the traditional Chinese medicine polysaccharides by the bacterial consortium of the present invention, the efficacy test of the traditional Chinese medicine polysaccharides and the bacterial composition of the present invention for promoting anti-inflammatory, the efficacy test of the traditional Chinese medicine polysaccharides and the bacterial composition of the present invention for improving chronic pulmonary obstructive disease, and the efficacy test of the traditional Chinese medicine polysaccharides and the bacterial composition of the present invention for improving obesity and metabolic syndrome to prove that the traditional Chinese medicine polysaccharides and the bacterial composition of the present invention can be applied to prepare compositions with corresponding efficacy.

### Example 1

### The Traditional Chinese Medicine Polysaccharides Promotes Growth of the Bacterial Consortium

In one embodiment of the present invention, in order to test that the traditional Chinese medicine polysaccharides of the present invention can indeed be utilized and digested by the bacterial consortium of the present invention, a minimal medium supplemented with the different traditional Chinese medicine polysaccharides of the present invention as a nutrient source was used to cultivate the bacterial consortium of the present invention in vitro to observe the growth of the bacterial consortium of the present invention.

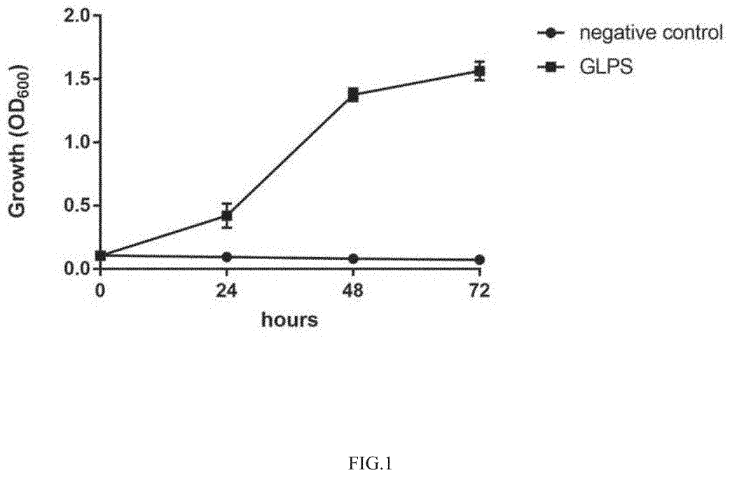

First, in order to cultivate the bacterial consortium of the present invention in the minimal medium, the P. goldsteinii MTS01 strain, the P. distasonis ATCC8503 strain, the B. xylanisolvens DSM18836 strain, the B. uniformis ATCC8492 strain, the B. fragilis NCTC9343 strain, and the B. ovatus ATCC8483 strain were cultured overnight by the aforementioned bacterial cultivation method of Bacteroidetes. The bacteria were pelleted by centrifugation and the supernatant was removed. The pellet was washed several times with sterile phosphate buffered saline (PBS), and then re-suspended with the minimal medium (the minimal medium contained per liter: 50 ml of Mineral 3B solution (contained per liter: KH₂PO₄, 18 g; NaCl, 18 g; CaCl₂. 2H₂O, 0.52 g; MgCl₂. 6H₂O, 0.4 g; MnCl₂.4H₂O, 0.2 g; CoCl₂. 6H₂O, 0.02 g; NH₄Cl, 10 g; and Na₂SO₄, 5 g.), 1 g L-cysteine, 10 mL of 50 mg/mL Hemin solution, 1 mL of 0.01% vitamin B12 solution, 1.5 mL of 10 mM FeSO₄ solution, and 20 mL of 10% NaHCO₃ solution). The six bacteria strains were mixed in equal proportions to a total of about 2×10⁷ CFU/mL to form the bacterial consortium of the invention. The bacterial consortium was then cultivated in the following three groups: (1) the minimal medium supplemented with 5 mg/mL of the Ganoderma lucidum polysaccharide (GLPS) of the present invention; (2) the minimum medium containing 5 mg/mL of the Dendrobium huoshanense polysaccharide (DHPS) of the present invention; and (3) the minimal medium supplemented with 5 mg/mL of the Hirsutella sinensis polysaccharide of the present invention. The starting time point of observation was when the optical density (OD600) of the cultures was 0.1 measured by a spectrophotometer (NanoPhotometer, Implen). At the 0th hour, 24th hour, 48th hour, and 72nd hour of the cultivation, the optical density (OD600) of each culture was measured respectively to observe the growth of the bacterial consortium of the present invention using the three different traditional Chinese medicine polysaccharides of the present invention as the nutrient source. The results were shown in FIGS. 1 to 3, respectively; wherein, the cultivation was performed under anaerobic conditions at 37° C., and a minimal medium only supplemented with water was used as a negative control. Data of the experimental results obtained were the mean±SD of three independent experiments.

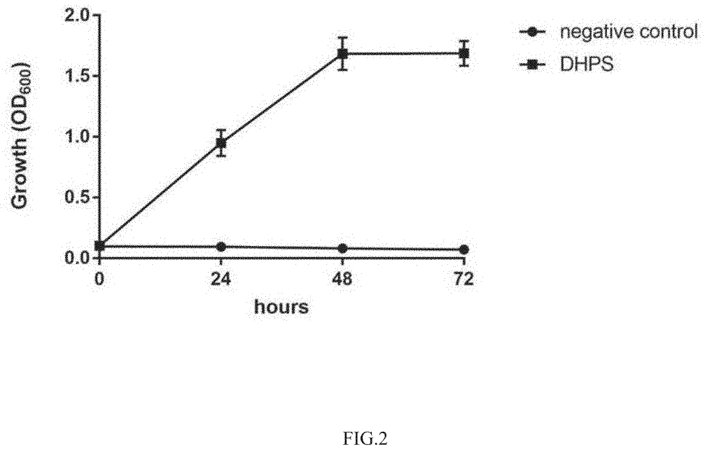

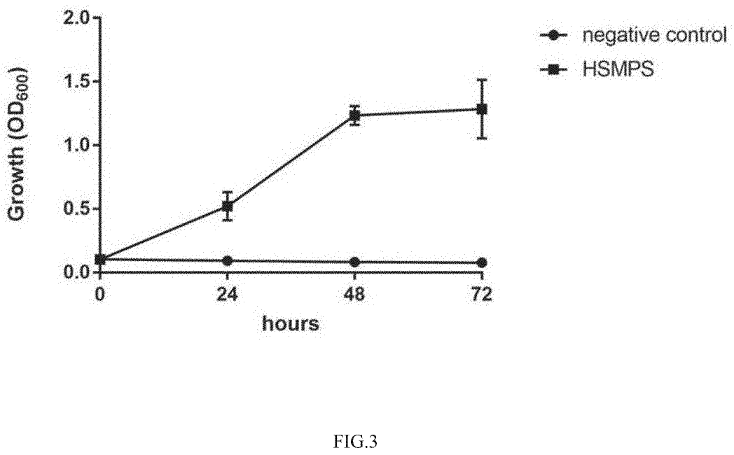

FIG. 1 was the growth curve of the bacterial consortium of the present invention over time in the minimal medium supplemented with the Ganoderma lucidum polysaccharide of the present invention; FIG. 2 was the growth curve of the bacterial consortium of the present invention over time in the minimal medium supplemented with the Dendrobium huoshanense polysaccharide of the present invention; and FIG. 3 was the growth curve of the bacterial consortium of the present invention over time in the minimal medium supplemented with the Hirsutella sinensis polysaccharide of the present invention. As shown in FIGS. 1 to 3, compared with the negative control group only supplemented with water, no matter using the Ganoderma lucidum polysaccharide, the Dendrobium huoshanense polysaccharide, or the Hirsutella sinensis polysaccharide of the present invention as the nutrient source, the growth of the bacterial consortium of the present invention in the minimal medium would be significantly promoted. The results indicated that the bacterial consortium of the present invention composed of Bacteroidetes can effectively utilize and digest traditional Chinese medicine polysaccharides of the present invention.

### Example 2

### The Traditional Chinese Medicine Polysaccharides Induces Secretion of Anti-Inflammatory Cytokines

In one embodiment of the present invention, in order to test the anti-inflammatory effect of the traditional Chinese medicine polysaccharides of the present invention and the bacterial composition of the present invention containing the traditional Chinese medicine polysaccharides, the traditional Chinese medicine polysaccharides of the present invention or the bacterial composition of the present invention containing the traditional Chinese medicine polysaccharides was used to stimulate macrophages, and the amount of interleukin 10 (IL-10) secreted into the culture medium was measured. IL-10 is a cytokine to inhibit the inflammatory responses.

In the embodiment of the present invention, the macrophages were murine macrophage RAW264.7 cell line. The murine macrophage cell line was purchased from ATCC and the number was ATCC® TIB-71™. The macrophages were cultured with DMEM (Dulbecco's Modified Eagle Medium, Gibco, USA) containing 10% fetal bovine serum, and 1% antibiotic-antimycotic (Thermo Fisher Scientific, USA, No. 12100-046) in a 24-well culture plate with 2×10⁵ cells per well at the 37° C. incubator containing 5% carbon dioxide for 24 hours, so that the macrophages could be attached to the bottom of the culture plate.

Further, the bacterial consortium used in the present invention was prepared as follow: the P. goldsteinii MTS01 strain, the P. distasonis ATCC8503 strain, the B. xylanisolvens DSM18836 strain, the B. uniformis ATCC8492 strain, the B. fragilis NCTC9343 strain, and the B. ovatus ATCC8483 strain were cultured overnight by the aforementioned bacterial cultivation method of Bacteroidetes. The bacteria were pelleted by centrifugation and the supernatant was removed. The pellet was washed several times with PBS, and then re-suspended with DMEM. The six bacteria strains were mixed in equal proportions to a total of about 2×10⁷ CFU/mL to form the bacterial consortium of the invention.

2-1 the Ganoderma lucidum Polysaccharide and the Bacterial Composition Containing the Ganoderma lucidum Polysaccharide Promote Secretion of Anti-Inflammatory Cytokines

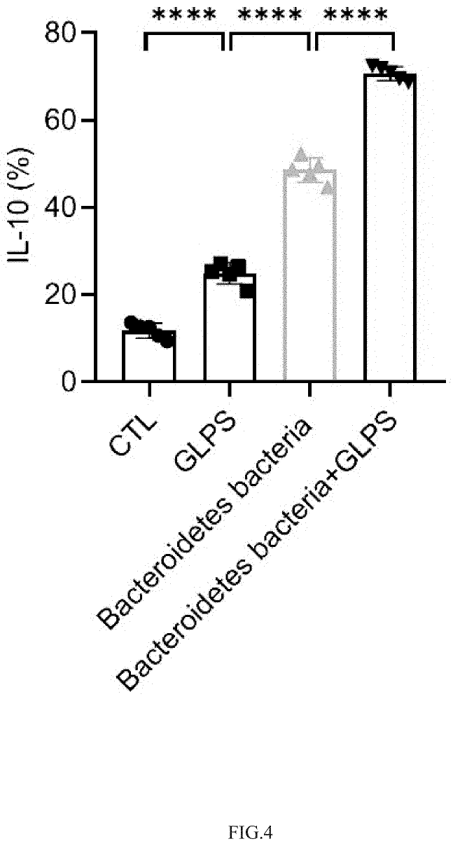

After the macrophages attached to the bottom of the culture plate, the macrophages were separated into the following four groups (n=5): (1) the control group (CTL): the macrophages were only treated with 10 μL of PBS for 6 hours; (2) the experimental group (GLPS): the macrophages were treated with 10 μL of the 2 mg/mL Ganoderma lucidum polysaccharide of the present invention (the total concentration was 200 μg/mL) for 6 hours; (3) the experimental group (Bacteroidetes bacteria): the macrophages were treated with 10 μL of the 2×10⁷ CFU/mL bacterial consortium of the present invention (the total amount was 2×10⁶ CFU) for 6 hours; and (4) the experimental group (Bacteroidetes bacteria+GLPS): the macrophages were treated with 10 μL of the bacterial composition of the present invention containing the Ganoderma lucidum polysaccharide (the total concentration of the Ganoderma lucidum polysaccharide was 200 μg/mL, and the total amount of the bacterial consortium was 2×10⁶ CFU) for 6 hours. Next, the same volume of supernatant of cell culture medium was taken out, and the secretion of IL-10 in each group was analyzed by Enzyme-linked immunosorbent assay (ELISA). The results were shown in FIG. 4. The secretion of IL-10 was defined as 100% when the amount thereof was 2000 pg/mL to convert the percentage of IL-10 secreted in each group. Data of the experimental results obtained were the mean±SD of five independent experiments. The Newman-Keuls multiple comparison post hoc one-way ANOVA analysis was used for statistical analysis; wherein, **** represented p-value<0.0001.

As shown in FIG. 4, compared with the macrophages only treated with PBS, after being treated with the Ganoderma lucidum polysaccharide of the present invention, the bacterial consortium of the present invention, or the bacterial composition of the present invention containing the Ganoderma lucidum polysaccharide, the IL-10 level secreted by the macrophages would significantly increase. The efficacy of the bacterial composition of the present invention containing the Ganoderma lucidum polysaccharide was significantly better than that of the Ganoderma lucidum polysaccharide or the bacterial consortium of the present invention alone. The results indicated that no matter the Ganoderma lucidum polysaccharide of the present invention, the bacterial consortium of the present invention, or the bacterial composition of the present invention containing the Ganoderma lucidum polysaccharide can effectively promote the secretion of anti-inflammatory cytokines to inhibit the inflammatory responses.

2-2 the Dendrobium huoshanense Polysaccharide and the Bacterial Composition Containing the Dendrobium huoshanense Polysaccharide Promote Secretion of Anti-Inflammatory Cytokines

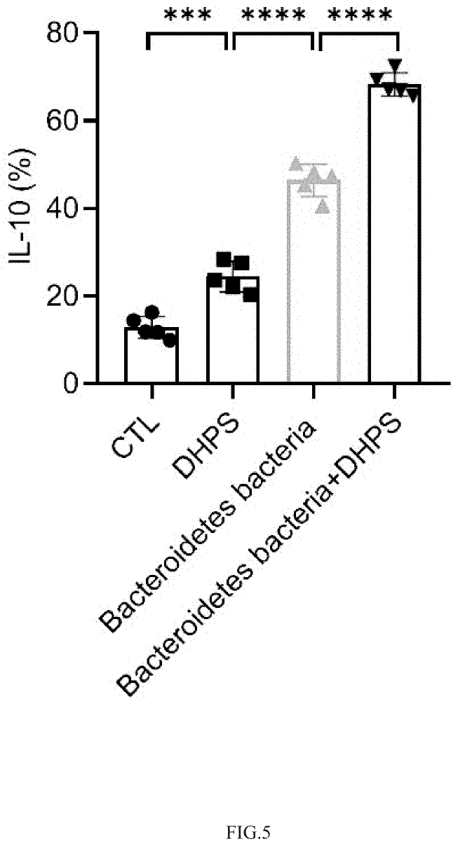

After the macrophages attached to the bottom of the culture plate, the macrophages were separated into the following four groups (n=5): (1) the control group (CTL): the macrophages were only treated with 10 μL of PBS for 6 hours; (2) the experimental group (DHPS): the macrophages were treated with 10 μL of the 2 mg/mL Dendrobium huoshanense polysaccharide of the present invention (the total concentration was 200 μg/mL) for 6 hours; (3) the experimental group (Bacteroidetes bacteria): the macrophages were treated with 10 μL of the 2×10⁷ CFU/mL bacterial consortium of the present invention (the total amount was 2×10⁶ CFU) for 6 hours; and (4) the experimental group (Bacteroidetes bacteria+DHPS): the macrophages were treated with 10 μL of the bacterial composition of the present invention containing the Dendrobium huoshanense polysaccharide (the total concentration of the Dendrobium huoshanense polysaccharide was 200 μg/mL, and the total amount of the bacterial consortium was 2×10⁶ CFU) for 6 hours. Next, the same volume of supernatant of cell culture medium was taken out, and the secretion of IL-10 in each group was analyzed by ELISA. The results were shown in FIG. 5. The secretion of IL-10 was defined as 100% when the amount thereof was 2000 pg/mL to convert the percentage of IL-10 secreted in each group. Data of the experimental results obtained were the mean±SD of five independent experiments. The Newman-Keuls multiple comparison post hoc one-way ANOVA analysis was used for statistical analysis; wherein, *** represented p-value<0.001, and **** represented p-value<0.0001.

As shown in FIG. 5, compared with the macrophages only treated with PBS, after being treated with the Dendrobium huoshanense polysaccharide of the present invention, the bacterial consortium of the present invention, or the bacterial composition of the present invention containing the Dendrobium huoshanense polysaccharide, the IL-10 level secreted by the macrophages would significantly increase. The efficacy of the bacterial composition of the present invention containing the Dendrobium huoshanense polysaccharide was significantly better than that of the Dendrobium huoshanense polysaccharide or the bacterial consortium of the present invention alone. The results indicated that no matter the Dendrobium huoshanense polysaccharide of the present invention, the bacterial consortium of the present invention, or the bacterial composition of the present invention containing the Dendrobium huoshanense polysaccharide can effectively promote the secretion of anti-inflammatory cytokines to inhibit the inflammatory responses.

2-3 the Hirsutella sinensis Polysaccharide and the Bacterial Composition Containing the Hirsutella sinensis Polysaccharide Promote Secretion of Anti-Inflammatory Cytokines

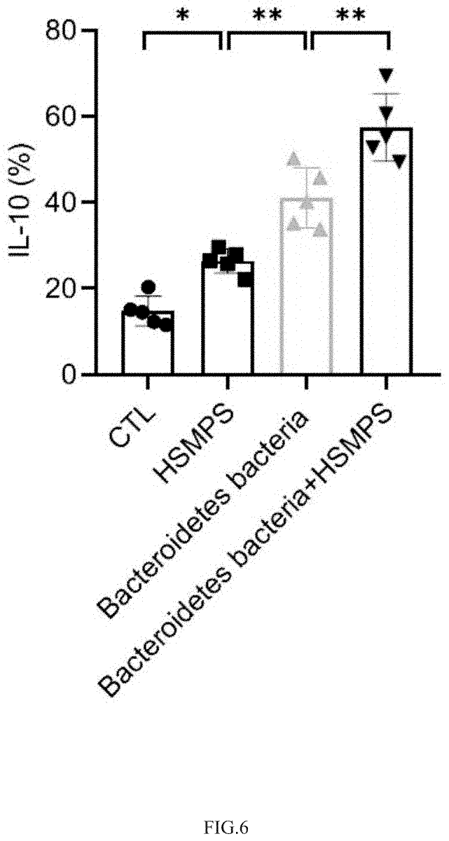

After the macrophages attached to the bottom of the culture plate, the macrophages were separated into the following four groups (n=5): (1) the control group (CTL): the macrophages were only treated with 10 μL of PBS for 6 hours; (2) the experimental group (HSMPS): the macrophages were treated with 10 μL of the 2 mg/mL Hirsutella sinensis polysaccharide of the present invention (the total concentration was 200 μg/mL) for 6 hours; (3) the experimental group (Bacteroidetes bacteria): the macrophages were treated with 10 μL of the 2×10⁷ CFU/mL bacterial consortium of the present invention (the total amount was 2×10⁶ CFU) for 6 hours; and (4) the experimental group (Bacteroidetes bacteria+HSMPS): the macrophages were treated with 10 μL of the bacterial composition of the present invention containing the Hirsutella sinensis polysaccharide (the total concentration of the Hirsutella sinensis polysaccharide was 200 μg/mL, and the total amount of the bacterial consortium was 2×10⁶ CFU) for 6 hours. Next, the same volume of supernatant of cell culture medium was taken out, and the secretion of IL-10 in each group was analyzed by ELISA. The results were shown in FIG. 6. The secretion of IL-10 was defined as 100% when the amount thereof was 2000 pg/mL to convert the percentage of IL-10 secreted in each group. Data of the experimental results obtained were the mean±SD of five independent experiments. The Newman-Keuls multiple comparison post hoc one-way ANOVA analysis was used for statistical analysis; wherein, * represented p-value<0.05, and ** represented p-value<0.01.

As shown in FIG. 6, compared with the macrophages only treated with PBS, after being treated with the Hirsutella sinensis polysaccharide of the present invention, the bacterial consortium of the present invention, or the bacterial composition of the present invention containing the vpolysaccharide, the IL-10 level secreted by the macrophages would significantly increase. The efficacy of the bacterial composition of the present invention containing the Hirsutella sinensis polysaccharide was significantly better than that of the Hirsutella sinensis polysaccharide or the bacterial consortium of the present invention alone. The results indicated that no matter the Hirsutella sinensis polysaccharide of the present invention, the bacterial consortium of the present invention, or the bacterial composition of the present invention containing the Hirsutella sinensis polysaccharide can effectively promote the secretion of anti-inflammatory cytokines to inhibit the inflammatory responses.

### Example 3

The Ganoderma lucidum Polysaccharide and the Bacterial Composition Containing the Ganoderma lucidum Polysaccharide Improve Chronic Obstructive Pulmonary Disease

In one embodiment of the present invention, in order to test the effects of the Ganoderma lucidum polysaccharide of the present invention and the bacterial composition of the present invention containing the Ganoderma lucidum polysaccharide on improving symptoms of chronic obstructive pulmonary disease (COPD) in a subject, mice with chronic obstructive pulmonary disease induced by cigarette smoke (cigarette smoke, CS) were used as an animal model for experiments.

In the embodiment of the present invention, animal experiments were approved by the Institutional Animal Care and Use Protocol of Fu Jen Catholic University and were performed according to their guidelines. The experimental animals used herein were 8-to-10-week-old C57BL/6 mice which were purchased from the National Laboratory Animal Center (NLAC, Taipei, Taiwan) and kept under sterile conditions, following a 12-hour light/dark cycle, and were with one-week acclimatization period under this condition. During the whole period of the experiments, the mice were fed with normal chow diet (LabDiet, USA, Laboratory Autoclavable Rodent Diet 5010).

In the embodiment of the present invention, the Ganoderma lucidum polysaccharide used in the animal experiments was prepared by adding 100 μL of PBS into 100 μL of the 5 mg/mL Ganoderma lucidum polysaccharide of the present invention.

In the embodiment of the present invention, the bacterial consortium used in the animal experiments was prepared as follow: the P. goldsteinii MTS01 strain, the P. distasonis ATCC8503 strain, the B. xylanisolvens DSM18836 strain, the B. uniformis ATCC8492 strain, the B. fragilis NCTC9343 strain, and the B. ovatus ATCC8483 strain were cultured overnight by the aforementioned bacterial cultivation method of Bacteroidetes. The bacteria were pelleted by centrifugation and the supernatant was removed. The pellet was washed several times with PBS, and then re-suspended with PBS. The six bacteria strains were mixed in equal proportions to a total of about 6×10⁸ CFU/100 μL (1×10⁸ CFU for each strain), and 100 μL of PBS was added.

In the embodiment of the present invention, the bacterial composition containing the Ganoderma lucidum polysaccharide used in the animal experiments was prepared by adding 100 μL of the 5 mg/mL Ganoderma lucidum polysaccharide of the present invention into the above-mentioned 6×10⁸ CFU/100 μL (1×10⁸ CFU for each strain) bacterial consortium.

Further, in the embodiment of the present invention, the Ganoderma lucidum polysaccharides, the bacterial consortium, and bacterial compositions containing the Ganoderma lucidum polysaccharides used in the animal experiments were all incubated in an anaerobic environment at 37° C. for 6 hours before being used to the experimental animals.

After the acclimatization period of the experimental mice was over, the 8-to-10-week-old C57BL/6 mice were separated into the following five groups (n=5): (1) the control group (CTL): the mice were exposed to indoor air, and were administered with 200 μL of PBS orally once a day five times a week for twelve weeks; (2) the comparative group (CS): the mice were exposed to cigarette smoke from twelve 3R4F cigarettes (Kentucky University) twice a day (twenty-four cigarettes per day) five times a week, and were administered with 200 μL of PBS orally once a day five times a week for twelve weeks; (3) the experimental group (CS+CLPS): the mice were exposed to cigarette smoke from twelve 3R4F cigarettes twice a day five times a week, and were administered with 200 μL of the above-mentioned Ganoderma lucidum polysaccharide of the present invention (25 mg/kg, about 0.5 mg per mouse) orally once a day five times a week for twelve weeks; (4) the experimental group (CS+Bacteroidetes bacteria): the mice were exposed to cigarette smoke from twelve 3R4F cigarettes twice a day five times a week, and were administered with 200 μL of the above-mentioned bacterial consortium of the present invention (3×10¹⁰ CFU/kg, about 6×10⁸ CFU per mouse) orally once a day five times a week for twelve weeks; and (5) the experimental group (CS+Bacteroidetes bacteria+CLPS): the mice were exposed to cigarette smoke from twelve 3R4F cigarettes twice a day five times a week, and were administered with 200 μL of the bacterial composition of the present invention containing the Ganoderma lucidum polysaccharide (25 mg/kg of the Ganoderma lucidum polysaccharide, about 0.5 mg per mouse; 3×10¹⁰ CFU/kg of bacterial consortium, about 6×10⁸ CFU per mouse) orally once a day five times a week for twelve weeks.

3-1 the Ganoderma lucidum Polysaccharide and the Bacterial Composition Containing the Ganoderma lucidum Polysaccharide Improve Body Weight Loss Caused by COPD

During the 12-week experimental duration, the percentage of body weight gain of each group of mice relative to the starting body weight in the 0th week was monitored every week, and the results were shown in FIG. 7A. Data of the experimental results obtained were the mean±SD of five independent experiments. The Newman-Keuls multiple comparison post hoc one-way ANOVA analysis was used for statistical analysis; wherein, ** represented p-value<0.01, *** represented p-value<0.001, and **** represented p-value<0.0001. Further, in the 12th week of the experiment, the percentage of body weight gain of each group of mice relative to the starting body weight in the 0th week was measured, and the results were shown in FIG. 7B. Data of the experimental results obtained were the mean±IQR of five independent experiments, and the Newman-Keuls multiple comparison post hoc one-way ANOVA analysis was used for statistical analysis; wherein, ** represents p value<0.01; *** represents p-value<0.001; and **** represents p-value<0.0001.

As shown in FIGS. 7A and 7B, compared with the mice of the control group exposed to indoor air, the percentage of body weight gain of the mice of the comparison group, in which COPD was induced by cigarette smoke, would significantly reduce and even be negative values, that is, the body weight of the mice of the comparison group would be lower than the starting body weight in the 0th week as the experiment progressed. However, after COPD was induced by cigarette smoke in the mice, compared with the comparison group, the administration of the Ganoderma lucidum polysaccharide of the present invention, the bacterial consortium of the present invention, or the bacterial composition of the present invention containing the Ganoderma lucidum polysaccharide caused the percentage of body weight gain of the mice significantly increase. The efficacy of the bacterial composition of the present invention containing the Ganoderma lucidum polysaccharide was significantly better than that of the Ganoderma lucidum polysaccharide or the bacterial consortium of the present invention alone. The results indicated that no matter the Ganoderma lucidum polysaccharide of the present invention, the bacterial consortium of the present invention, or the bacterial composition of the present invention containing the Ganoderma lucidum polysaccharide can effectively improve the problem of body weight loss in subjects caused by COPD. In the bacterial composition of the present invention, after the Ganoderma lucidum polysaccharide was utilized and digested by the bacterial consortium, the active substances produced provided significantly improved effects.

3-2 the Ganoderma lucidum Polysaccharide and the Bacterial Composition Containing the Ganoderma lucidum Polysaccharide Improve Abnormal Lung Histopathology Caused by COPD

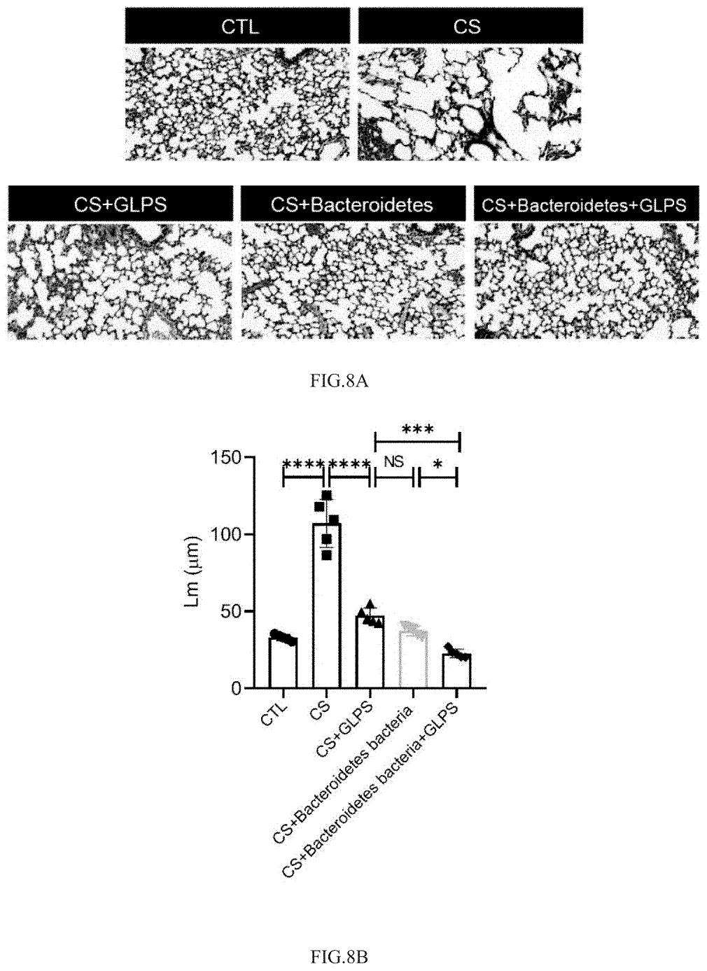

In one embodiment of the present invention, in order to more directly observe whether the Ganoderma lucidum polysaccharide of the present invention and the bacterial composition of the present invention containing Ganoderma lucidum polysaccharide can improve the abnormal histopathology of subjects with chronic obstructive pulmonary disease, the mice of the aforementioned 12-week experiments were sacrificed, and the lung tissues of each group of mice were taken out and fixed with formalin and then embedded in paraffin. The tissue sections with 4 mm of thickness were prepared and stained with hematoxylin and eosin (H&E). The stained sections were examined and recorded under a light microscope (Olympus, Tokyo, Japan), and the results were shown in FIG. 8A. The histological images were further analyzed using the ImageJ software (National Institutes of Health, Bethesda, USA) to determine the linear intercept (represented as Lm in FIG. 8B) to quantify the severity of abnormal lung histopathology and expressed in μm, wherein two randomly-selected fields from 10-15 sections of each group of mice were analyzed, and the results were shown in FIG. 8B. Data of the experimental results obtained were the mean±IQR of five independent experiments, and the Newman-Keuls multiple comparison post hoc one-way ANOVA analysis was used for statistical analysis; wherein, * represents p value<0.05; *** represents p-value<0.001; **** represents p-value<0.0001, and NS represented not significant.

As shown in FIGS. 8A and 8B, compared with the mice of the control group exposed to indoor air, the alveolar wall of the mice of the comparison group, in which COPD was induced by cigarette smoke, was seriously damaged and the air gap of the alveolar was also enlarged, indicating that the mice of the comparison group had abnormal lung histopathology such as emphysema. However, after COPD was induced by cigarette smoke in the mice, compared with the comparison group, the administration of the Ganoderma lucidum polysaccharide of the present invention, the bacterial consortium of the present invention, or the bacterial composition of the present invention containing the Ganoderma lucidum polysaccharide caused such abnormal lung histopathology of the mice significantly relieve to be closer to that of the mice of the control group. The efficacy of the bacterial composition of the present invention containing the Ganoderma lucidum polysaccharide was significantly better than that of the Ganoderma lucidum polysaccharide or the bacterial consortium of the present invention alone. The results indicated that no matter the Ganoderma lucidum polysaccharide of the present invention, the bacterial consortium of the present invention, or the bacterial composition of the present invention containing the Ganoderma lucidum polysaccharide can effectively improve the abnormal lung histopathology in subjects with COPD. In the bacterial composition of the present invention, after the Ganoderma lucidum polysaccharide was utilized and digested by the bacterial consortium, the active substances produced provided significantly improved effects.

3-3 the Ganoderma lucidum Polysaccharide and the Bacterial Composition Containing the Ganoderma lucidum Polysaccharide Improve Abnormal Lung Function Caused by COPD

In one embodiment of the present invention, in order to further observe whether the Ganoderma lucidum polysaccharide of the present invention and the bacterial composition of the present invention containing Ganoderma lucidum polysaccharide can improve the abnormal lung function of subjects with chronic obstructive pulmonary disease, all of the mice of the aforementioned 12-week experiments were anesthetized, tracheostomized, and placed in a forced pulmonary maneuver system (Buxco Research Systems, USA, hereinafter referred to as Buxco system) for evaluating lung functions. First, the anesthetized mice were induced to have 100 breaths/min on average and the Buxco system was used to perform three semi-automatic maneuvers on the mice, including the determination of functional residual capacity (FRC) determined by Boyle's law, quasistatic P-V, and fast flow volume maneuver. The operation for quasistatic P-V was to measure chord compliance (Cchord). The operation for fast flow volume maneuver was to record forced expiratory volume (FEV), including the forced vital capacity (FVC) and the forced expiratory volume at the 100th millisecond (FEV100). The operation for fast flow drive was to record the forced expiratory volume (Forced expiratory volume, FEV), including the forced vital capacity (FVC) and the forced expiratory volume at the 100th millisecond (FEV100).

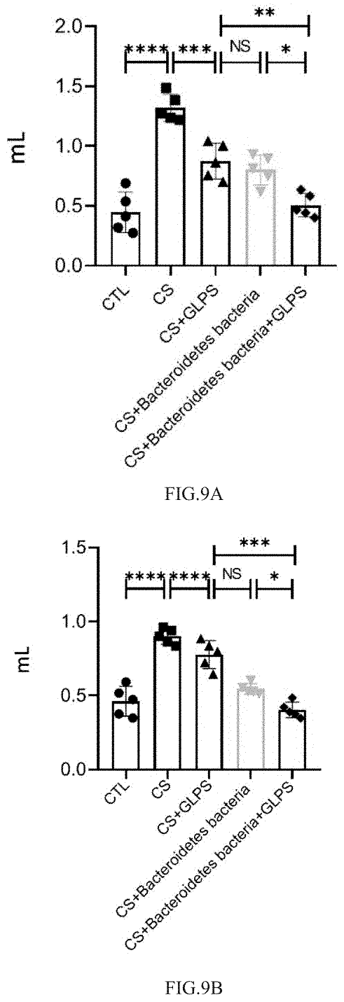

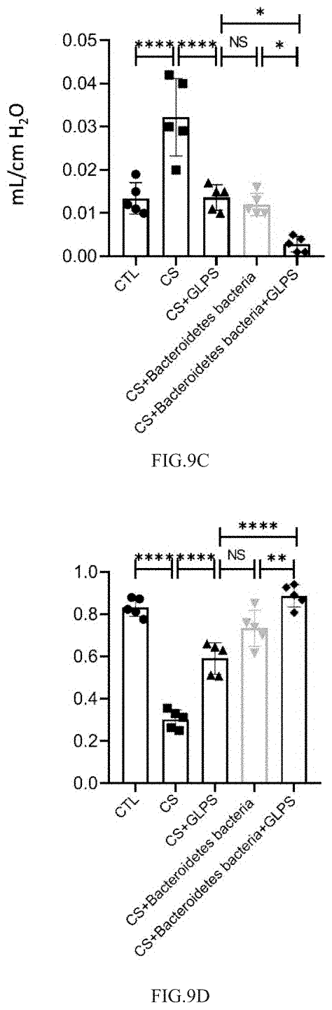

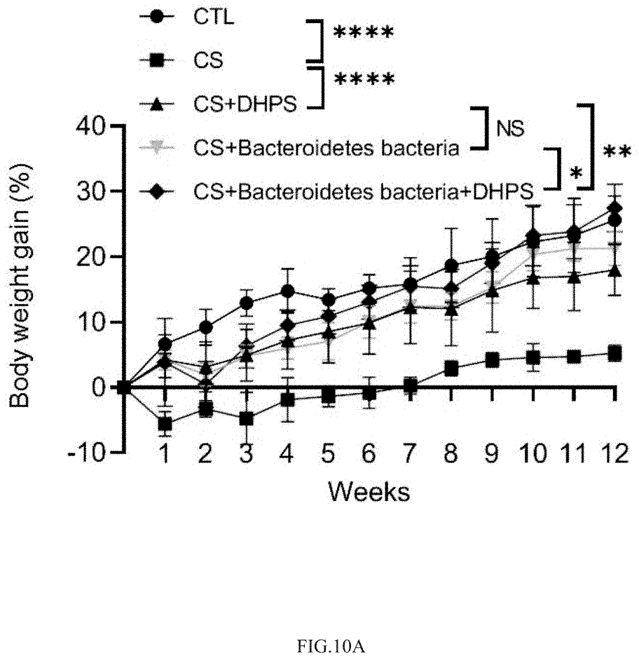

The results of the Ganoderma lucidum polysaccharide of the present invention and the bacterial composition of the present invention containing Ganoderma lucidum polysaccharide on improving abnormality of FRC in the subjects with COPD were shown in FIG. 9A, wherein FRC was expressed in the unit of mL; the results on improving abnormality of FVC in the subjects with COPD were shown in FIG. 9B, wherein FVC was expressed in the unit of mL; the results on improving abnormality of Cchord in the subjects with COPD were shown in FIG. 9C, wherein COPD was expressed in the unit of mL/cm H₂O; and the results on improving abnormality of FEV100/FVC ration in the subjects with COPD were shown in FIG. 9D. All the above maneuvers and perturbations were continuously performed until three correct measurements were achieved. The average of the three measurements of the above parameters for each mouse in each group was used as the result value for that parameter for that group of mice. Data of the experimental results obtained were the mean±IQR of five independent experiments, and the Newman-Keuls multiple comparison post hoc one-way ANOVA analysis was used for statistical analysis; wherein, * represents p value<0.05; ** represents p-value<0.01; *** represents p-value<0.001; **** represents p-value<0.0001, and NS represented not significant.

As shown in FIGS. 9A to 9D, compared with the mice of the control group exposed to indoor air, the FRC and Cchord of the mice of the comparison group, in which COPD was induced by cigarette smoke, significantly increased, indicating that the mice with emphysema induced by cigarette smoke had hyperinflation. Furthermore, since the mice of the comparative group had a larger lung volume during maximum inflation, the FVC thereof also significantly increased under forced exhalation. The index of airflow obstruction during expiration, i.e. FEV100/FVC ratio, of the mice of the comparison group significantly decreased. The results indicated that the induction of COPD in mice by cigarette smoke would indeed reduce the lung function of the mice.

However, after COPD was induced by cigarette smoke in the mice, compared with the comparison group, the administration of the Ganoderma lucidum polysaccharide of the present invention, the bacterial consortium of the present invention, or the bacterial composition of the present invention containing the Ganoderma lucidum polysaccharide caused FRC, FVC, and Cchord significantly reduce and FEV100/FVC ration significantly increase. The efficacy of the bacterial composition of the present invention containing the Ganoderma lucidum polysaccharide was significantly better than that of the Ganoderma lucidum polysaccharide or the bacterial consortium of the present invention alone. The results indicated that no matter the Ganoderma lucidum polysaccharide of the present invention, the bacterial consortium of the present invention, or the bacterial composition of the present invention containing the Ganoderma lucidum polysaccharide can effectively improve the emphysema in subjects with COPD, and effectively improve the lung function of the subjects with COPD. In the bacterial composition of the present invention, after the Ganoderma lucidum polysaccharide was utilized and digested by the bacterial consortium, the active substances produced provided significantly improved effects.

### Example 4

The Dendrobium huoshanense Polysaccharide and the Bacterial Composition Containing the Dendrobium huoshanense Polysaccharide Improve Chronic Obstructive Pulmonary Disease

In one embodiment of the present invention, in order to test the effects of the Dendrobium huoshanense polysaccharide of the present invention and the bacterial composition of the present invention containing the Dendrobium huoshanense polysaccharide on improving symptoms of COPD in a subject, mice with chronic obstructive pulmonary disease induced by cigarette smoke were also used as an animal model for experiments.

In the embodiment of the present invention, the experimental animals used herein were also 8-to-10-week-old C57BL/6 mice, and the mice were kept under the conditions described in Example 3.

In the embodiment of the present invention, the Dendrobium huoshanense polysaccharide of used in the animal experiments was prepared by adding 100 μL of PBS into 100 μL of the 5 mg/mL Dendrobium huoshanense polysaccharide of the present invention.

In the embodiment of the present invention, the bacterial consortium used in the animal experiment was prepared as described in Example 3. The bacterial composition containing the Dendrobium huoshanense polysaccharide used in the animal experiment was prepared by adding 100 μL of the 5 mg/mL Dendrobium huoshanense polysaccharide of the present invention into the 6×10⁸ CFU/100 μL (1×10⁸ CFU for each strain) bacterial consortium described in Example 3.

Further, in the embodiment of the present invention, the Dendrobium huoshanense polysaccharides, the bacterial consortium, and bacterial compositions containing the Dendrobium huoshanense polysaccharides used in the animal experiments were all incubated in an anaerobic environment at 37° C. for 6 hours before being used to the experimental animals.

After the acclimatization period of the experimental mice was over, the 8-to-10-week-old C57BL/6 mice were separated into the following five groups (n=5): (1) the control group (CTL): the mice were exposed to indoor air, and were administered with 200 μL of PBS orally once a day five times a week for twelve weeks; (2) the comparative group (CS): the mice were exposed to cigarette smoke from twelve 3R4F cigarettes twice a day five times a week, and were administered with 200 μL of PBS orally once a day five times a week for twelve weeks; (3) the experimental group (CS+DHPS): the mice were exposed to cigarette smoke from twelve 3R4F cigarettes twice a day five times a week, and were administered with 200 μL of the above-mentioned Dendrobium huoshanense polysaccharide of the present invention (25 mg/kg, about 0.5 mg per mouse) orally once a day five times a week for twelve weeks; (4) the experimental group (CS+Bacteroidetes bacteria): the mice were exposed to cigarette smoke from twelve 3R4F cigarettes twice a day five times a week, and were administered with 200 μL of the above-mentioned bacterial consortium of the present invention (3×10¹⁰ CFU/kg, about 6×10⁸ CFU per mouse) orally once a day five times a week for twelve weeks; and (5) the experimental group (CS+Bacteroidetes bacteria+DHPS): the mice were exposed to cigarette smoke from twelve 3R4F cigarettes twice a day five times a week, and were administered with 200 μL of the bacterial composition of the present invention containing the Dendrobium huoshanense polysaccharide (25 mg/kg of the Dendrobium huoshanense polysaccharide, about 0.5 mg per mouse; 3×10¹⁰ CFU/kg of bacterial consortium, about 6×10⁸ CFU per mouse) orally once a day five times a week for twelve weeks. 4-1 The Dendrobium huoshanense polysaccharide and the bacterial composition containing the Dendrobium huoshanense polysaccharide improve body weight loss caused by COPD

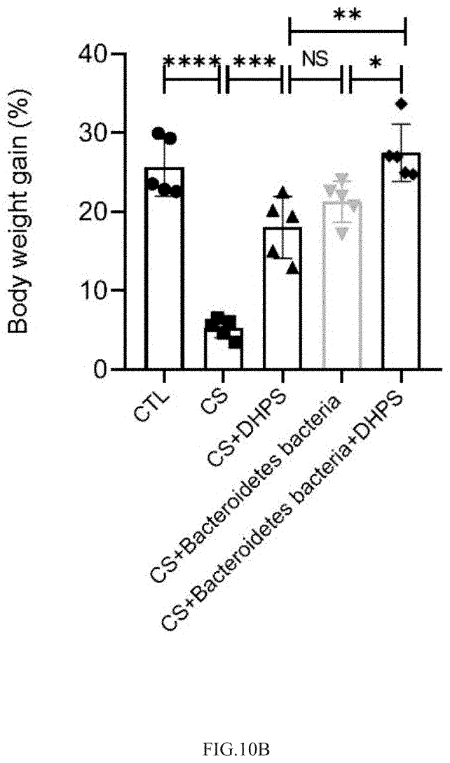

During the 12-week experimental duration, the percentage of body weight gain of each group of mice relative to the starting body weight in the 0th week was monitored every week, and the results were shown in FIG. 10A. Further, in the 12th week of the experiment, the percentage of body weight gain of each group of mice relative to the starting body weight in the 0th week was measured, and the results were shown in FIG. 10B. Data of the experimental results obtained were the mean±SD of five independent experiments, and the Newman-Keuls multiple comparison post hoc one-way ANOVA analysis was used for statistical analysis; wherein, * represents p value<0.05; ** represents p value<0.01; *** represents p-value<0.001; **** represents p-value<0.0001, and NS represented not significant.

As shown in FIGS. 10A and 10B, after COPD was induced by cigarette smoke in the mice, compared with the comparison group, the administration of the Dendrobium huoshanense polysaccharide of the present invention, the bacterial consortium of the present invention, or the bacterial composition of the present invention containing the Dendrobium huoshanense polysaccharide caused the percentage of body weight gain of the mice significantly increase. The efficacy of the bacterial composition of the present invention containing the Dendrobium huoshanense polysaccharide was significantly better than that of the Dendrobium huoshanense polysaccharide or the bacterial consortium of the present invention alone. The results indicated that no matter the Dendrobium huoshanense polysaccharide of the present invention, the bacterial consortium of the present invention, or the bacterial composition of the present invention containing the Dendrobium huoshanense polysaccharide can effectively improve the problem of body weight loss in subjects caused by COPD. In the bacterial composition of the present invention, after the Dendrobium huoshanense polysaccharide was utilized and digested by the bacterial consortium, the active substances produced provided significantly improved effects.

4-2 the Dendrobium huoshanense Polysaccharide and the Bacterial Composition Containing the Dendrobium huoshanense Polysaccharide Improve Abnormal Lung Histopathology Caused by COPD

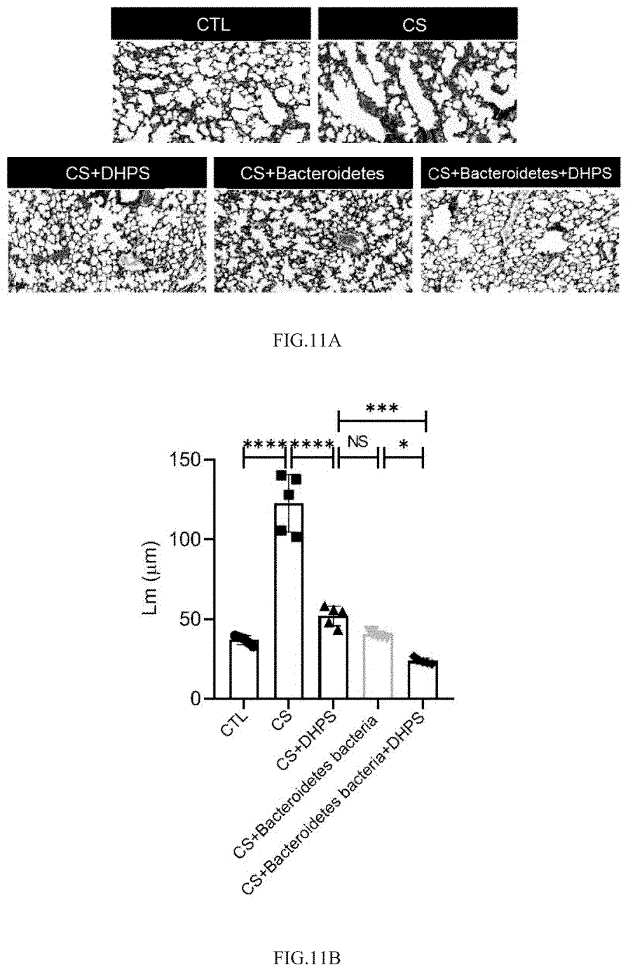

In one embodiment of the present invention, in order to more directly observe whether the Dendrobium huoshanense polysaccharide of the present invention and the bacterial composition of the present invention containing Dendrobium huoshanense polysaccharide can improve the abnormal histopathology of subjects with chronic obstructive pulmonary disease, the mice of the aforementioned 12-week experiments were sacrificed, and the lung tissues of each group of mice were taken out. The tissue sections were prepared and stained as the description in Example 3. The stained sections were examined and recorded under a light microscope (Olympus, Tokyo, Japan), and the results were shown in FIG. 11A. The histological images were also further analyzed using the ImageJ software (National Institutes of Health, Bethesda, USA) to determine the linear intercept (represented as Lm in FIG. 11B) to quantify the severity of abnormal lung histopathology and expressed in μm, and the results were shown in FIG. 11B. Data of the experimental results obtained were the mean±IQR of five independent experiments, and the Newman-Keuls multiple comparison post hoc one-way ANOVA analysis was used for statistical analysis; wherein, * represents p value<0.05; *** represents p-value<0.001; **** represents p-value<0.0001, and NS represented not significant.

As shown in FIGS. 11A and 11B, after COPD was induced by cigarette smoke in the mice, compared with the comparison group, the administration of the Dendrobium huoshanense polysaccharide of the present invention, the bacterial consortium of the present invention, or the bacterial composition of the present invention containing the Dendrobium huoshanense polysaccharide caused the abnormal lung histopathology of the mice significantly relieve to be closer to that of the mice of the control group. The efficacy of the bacterial composition of the present invention containing the Dendrobium huoshanense polysaccharide was significantly better than that of the Dendrobium huoshanense polysaccharide or the bacterial consortium of the present invention alone. The results indicated that no matter the Dendrobium huoshanense polysaccharide of the present invention, the bacterial consortium of the present invention, or the bacterial composition of the present invention containing the Dendrobium huoshanense polysaccharide can effectively improve the abnormal lung histopathology in subjects with COPD. In the bacterial composition of the present invention, after the Dendrobium huoshanense polysaccharide was utilized and digested by the bacterial consortium, the active substances produced provided significantly improved effects.

4-3 the Dendrobium huoshanense Polysaccharide and the Bacterial Composition Containing the Dendrobium huoshanense Polysaccharide Improve Abnormal Lung Function Caused by COPD

In one embodiment of the present invention, in order to further observe whether the Dendrobium huoshanense polysaccharide of the present invention and the bacterial composition of the present invention containing Dendrobium huoshanense polysaccharide can improve the abnormal lung function of subjects with chronic obstructive pulmonary disease, all of the mice of the aforementioned 12-week experiments were evaluated lung functions according to the method described in Example 3.

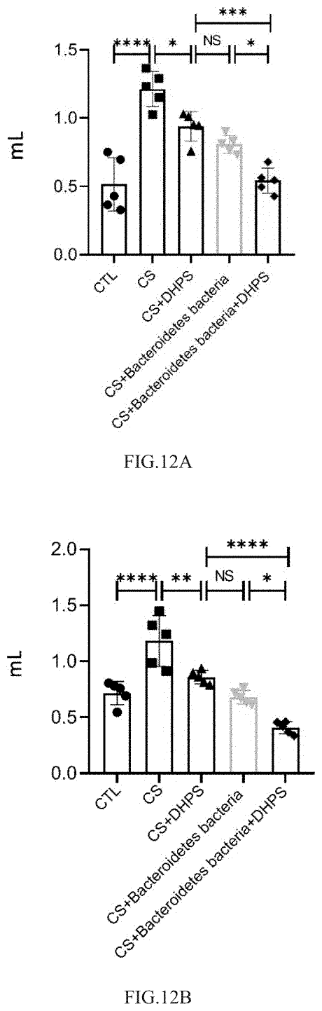

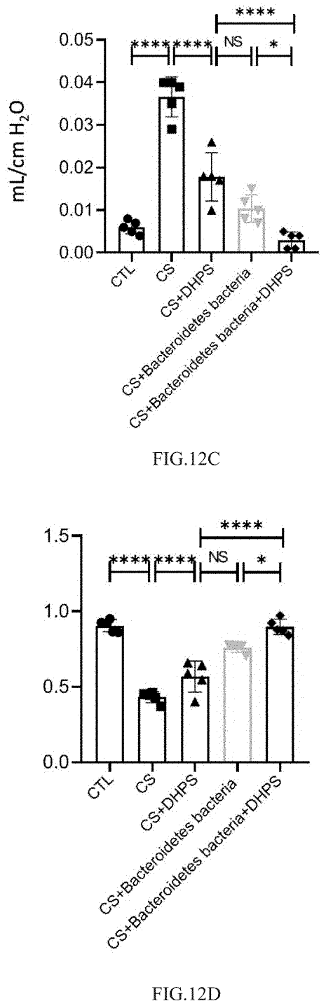

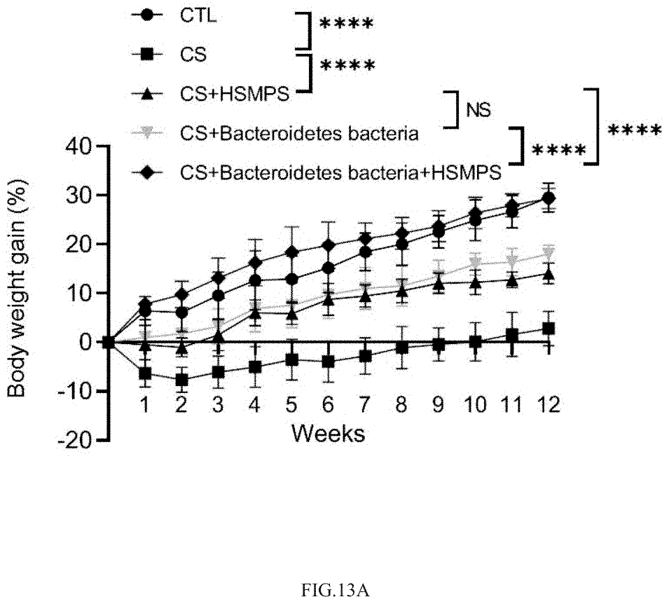

The results of the Dendrobium huoshanense polysaccharide of the present invention and the bacterial composition of the present invention containing Dendrobium huoshanense polysaccharide on improving abnormality of FRC in the subjects with COPD were shown in FIG. 12A, wherein FRC was expressed in the unit of mL; the results on improving abnormality of FVC in the subjects with COPD were shown in FIG. 12B, wherein FVC was expressed in the unit of mL; the results on improving abnormality of Cchord in the subjects with COPD were shown in FIG. 12C, wherein COPD was expressed in the unit of mL/cm H₂O; and the results on improving abnormality of FEV100/FVC ration in the subjects with COPD were shown in FIG. 12D. All the above maneuvers and perturbations were continuously performed until three correct measurements were achieved. The average of the three measurements of the above parameters for each mouse in each group was used as the result value for that parameter for that group of mice. Data of the experimental results obtained were the mean±IQR of five independent experiments, and the Newman-Keuls multiple comparison post hoc one-way ANOVA analysis was used for statistical analysis; wherein, * represents p value<0.05; ** represents p-value<0.01; *** represents p-value<0.001; **** represents p-value<0.0001, and NS represented not significant.

As shown in FIGS. 12A to 12D, after COPD was induced by cigarette smoke in the mice, compared with the comparison group, the administration of the Dendrobium huoshanense polysaccharide of the present invention, the bacterial consortium of the present invention, or the bacterial composition of the present invention containing the Dendrobium huoshanense polysaccharide caused FRC, FVC, and Cchord significantly reduce and FEV100/FVC ration significantly increase. The efficacy of the bacterial composition of the present invention containing the Dendrobium huoshanense polysaccharide was significantly better than that of the Dendrobium huoshanense polysaccharide or the bacterial consortium of the present invention alone. The results indicated that no matter the Dendrobium huoshanense polysaccharide of the present invention, the bacterial consortium of the present invention, or the bacterial composition of the present invention containing the Dendrobium huoshanense polysaccharide can effectively improve the emphysema in subjects with COPD, and effectively improve the lung function of the subjects with COPD. In the bacterial composition of the present invention, after the Dendrobium huoshanense polysaccharide was utilized and digested by the bacterial consortium, the active substances produced provided significantly improved effects.

### Example 5

The Hirsutella sinensis Polysaccharide and the Bacterial Composition Containing the Hirsutella sinensis Polysaccharide Improve Chronic Obstructive Pulmonary Disease

In one embodiment of the present invention, in order to test the effects of the Hirsutella sinensis polysaccharide of the present invention and the bacterial composition of the present invention containing the Hirsutella sinensis polysaccharide on improving symptoms of COPD in a subject, mice with chronic obstructive pulmonary disease induced by cigarette smoke were also used as an animal model for experiments.

In the embodiment of the present invention, the experimental animals used herein were also 8-to-10-week-old C57BL/6 mice, and the mice were kept under the conditions described in Example 3.

In the embodiment of the present invention, the Hirsutella sinensis polysaccharide of used in the animal experiments was prepared by adding 100 μL of PBS into 100 μL of the 5 mg/mL Hirsutella sinensis polysaccharide of the present invention.

In the embodiment of the present invention, the bacterial consortium used in the animal experiment was prepared as described in Example 3. The bacterial composition containing the Hirsutella sinensis polysaccharide used in the animal experiment was prepared by adding 100 μL of the 5 mg/mL Hirsutella sinensis polysaccharide of the present invention into the 6×10⁸ CFU/100 μL (1×10⁸ CFU for each strain) bacterial consortium described in Example 3.

Further, in the embodiment of the present invention, the Hirsutella sinensis polysaccharides, the bacterial consortium, and bacterial compositions containing the Hirsutella sinensis polysaccharides used in the animal experiments were all incubated in an anaerobic environment at 37° C. for 6 hours before being used to the experimental animals.

After the acclimatization period of the experimental mice was over, the 8-to-10-week-old C57BL/6 mice were separated into the following five groups (n=5): (1) the control group (CTL): the mice were exposed to indoor air, and were administered with 200 μL of PBS orally once a day five times a week for twelve weeks; (2) the comparative group (CS): the mice were exposed to cigarette smoke from twelve 3R4F cigarettes twice a day five times a week, and were administered with 200 μL of PBS orally once a day five times a week for twelve weeks; (3) the experimental group (CS+HSMPS): the mice were exposed to cigarette smoke from twelve 3R4F cigarettes twice a day five times a week, and were administered with 200 μL of the above-mentioned Hirsutella sinensis polysaccharide of the present invention (25 mg/kg, about 0.5 mg per mouse) orally once a day five times a week for twelve weeks; (4) the experimental group (CS+Bacteroidetes bacteria): the mice were exposed to cigarette smoke from twelve 3R4F cigarettes twice a day five times a week, and were administered with 200 μL of the above-mentioned bacterial consortium of the present invention (3×10¹⁰ CFU/kg, about 6×10⁸ CFU per mouse) orally once a day five times a week for twelve weeks; and (5) the experimental group (CS+Bacteroidetes bacteria+HSMPS): the mice were exposed to cigarette smoke from twelve 3R4F cigarettes twice a day five times a week, and were administered with 200 μL of the bacterial composition of the present invention containing the Hirsutella sinensis polysaccharide (25 mg/kg of the Hirsutella sinensis polysaccharide, about 0.5 mg per mouse; 3×10¹⁰ CFU/kg of bacterial consortium, about 6×10⁸ CFU per mouse) orally once a day five times a week for twelve weeks.

5-1 the Hirsutella sinensis Polysaccharide and the Bacterial Composition Containing the Hirsutella sinensis Polysaccharide Improve Body Weight Loss Caused by COPD

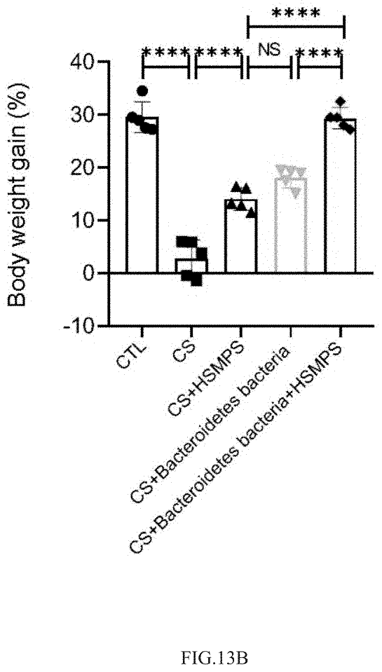

During the 12-week experimental duration, the percentage of body weight gain of each group of mice relative to the starting body weight in the 0th week was monitored every week, and the results were shown in FIG. 13A. Further, in the 12th week of the experiment, the percentage of body weight gain of each group of mice relative to the starting body weight in the 0th week was measured, and the results were shown in FIG. 13B. Data of the experimental results obtained were the mean±SD of five independent experiments, and the Newman-Keuls multiple comparison post hoc one-way ANOVA analysis was used for statistical analysis; wherein, **** represents p-value<0.0001, and NS represented not significant.

As shown in FIGS. 13A and 13B, after COPD was induced by cigarette smoke in the mice, compared with the comparison group, the administration of the Hirsutella sinensis polysaccharide of the present invention, the bacterial consortium of the present invention, or the bacterial composition of the present invention containing the Hirsutella sinensis polysaccharide caused the percentage of body weight gain of the mice significantly increase. The efficacy of the bacterial composition of the present invention containing the Hirsutella sinensis polysaccharide was significantly better than that of the Hirsutella sinensis polysaccharide or the bacterial consortium of the present invention alone. The results indicated that no matter the Hirsutella sinensis polysaccharide of the present invention, the bacterial consortium of the present invention, or the bacterial composition of the present invention containing the Hirsutella sinensis polysaccharide can effectively improve the problem of body weight loss in subjects caused by COPD. In the bacterial composition of the present invention, after the Hirsutella sinensis polysaccharide was utilized and digested by the bacterial consortium, the active substances produced provided significantly improved effects.

5-2 the Hirsutella sinensis Polysaccharide and the Bacterial Composition Containing the Hirsutella sinensis Polysaccharide Improve Abnormal Lung Histopathology Caused by COPD

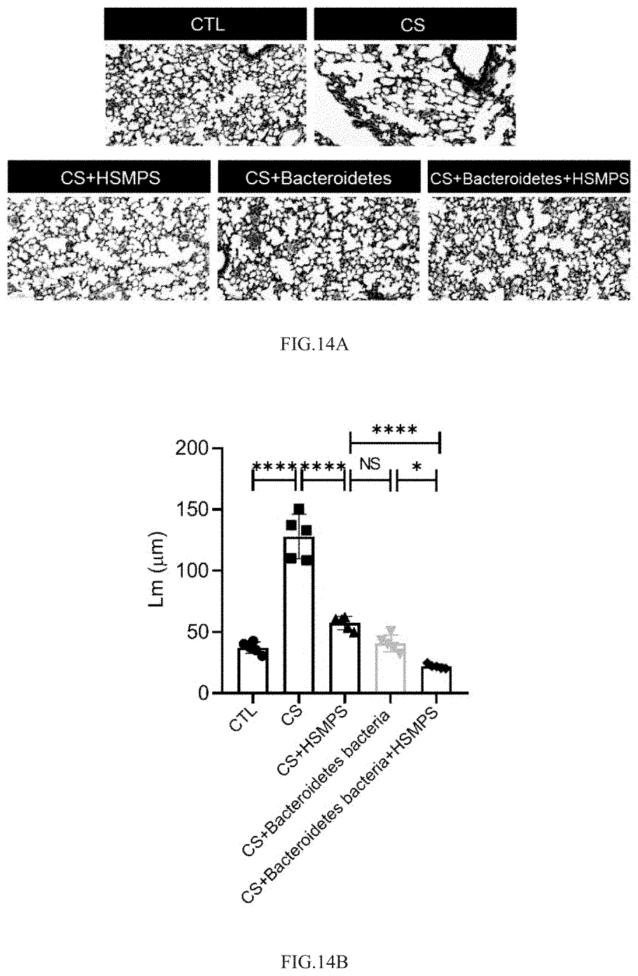

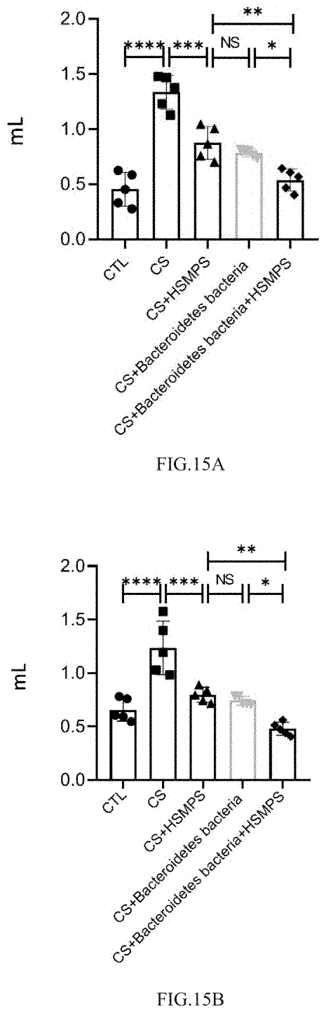

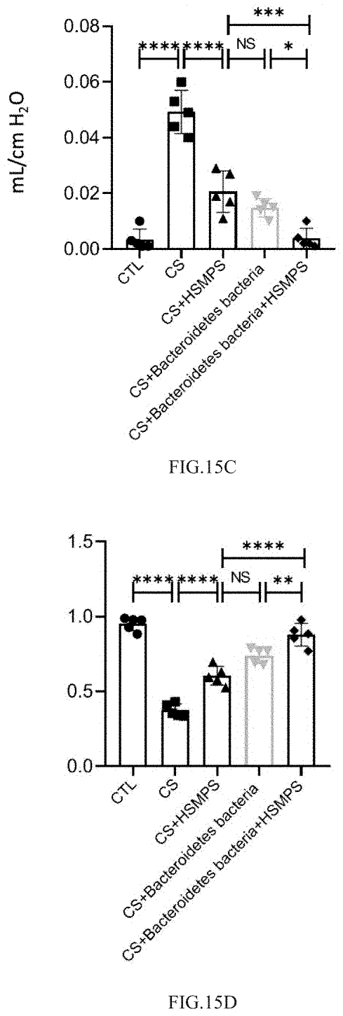

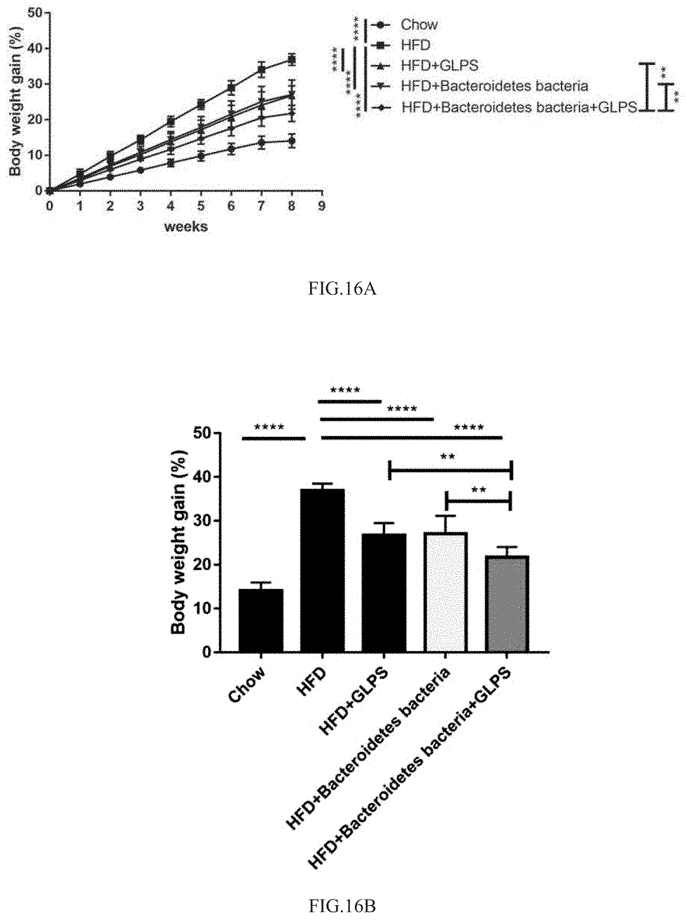

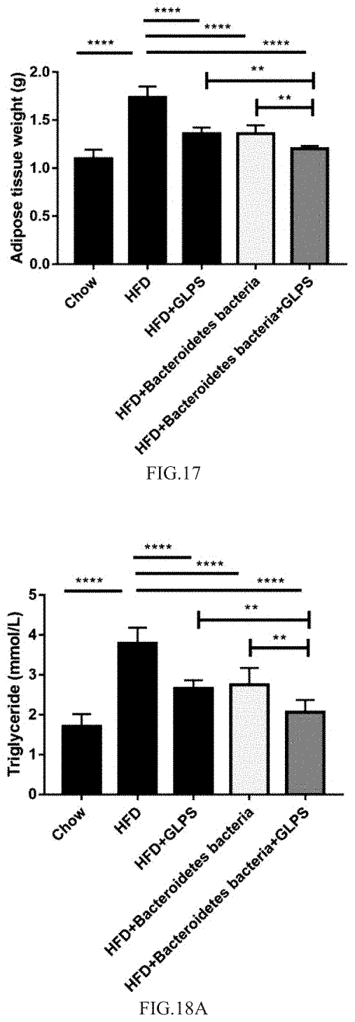

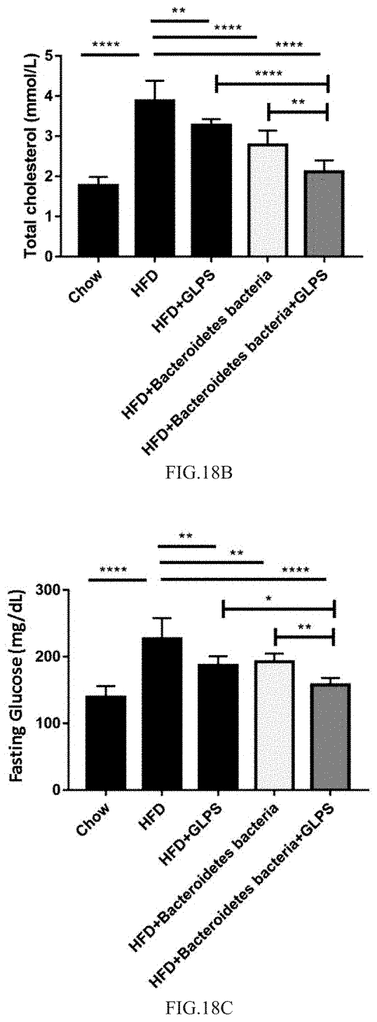

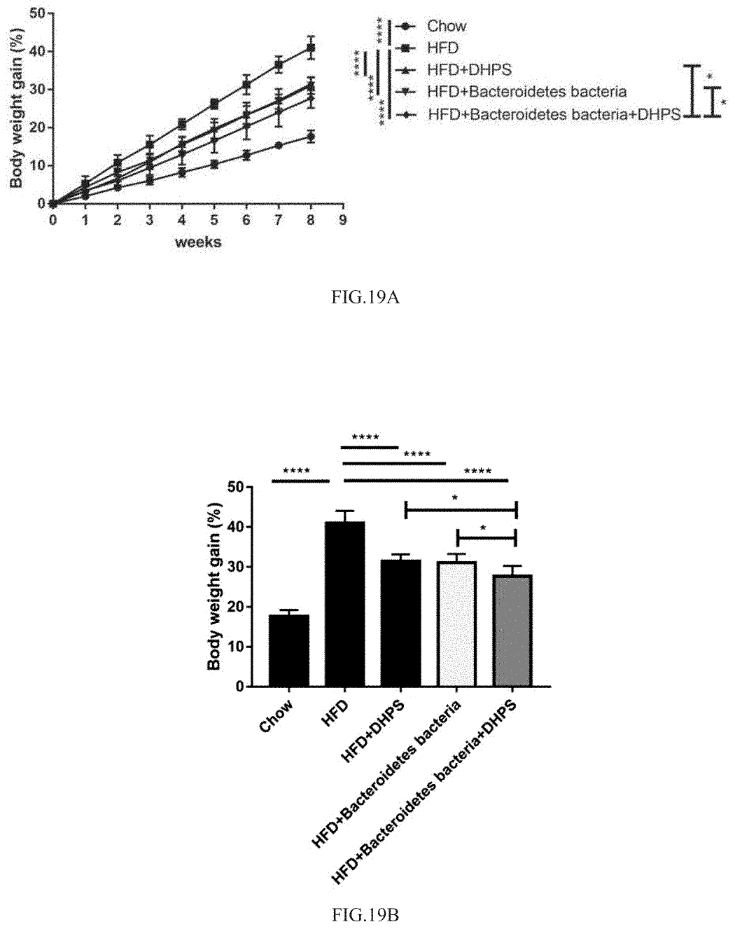

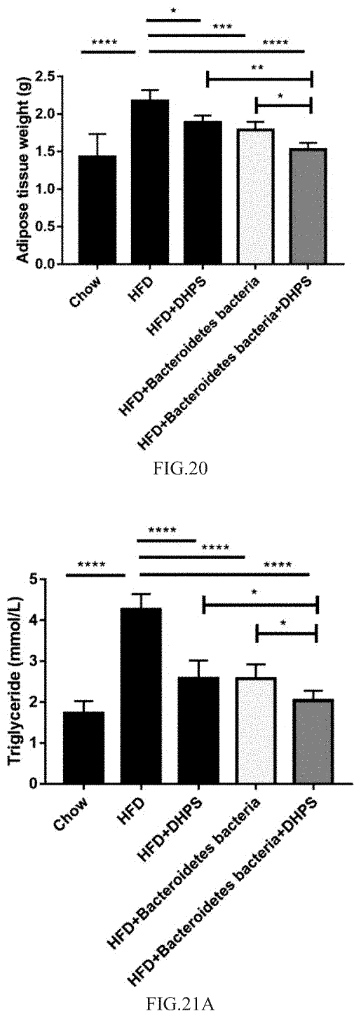

In one embodiment of the present invention, in order to more directly observe whether the Hirsutella sinensis polysaccharide of the present invention and the bacterial composition of the present invention containing Hirsutella sinensis polysaccharide can improve the abnormal histopathology of subjects with chronic obstructive pulmonary disease, the mice of the aforementioned 12-week experiments were sacrificed, and the lung tissues of each group of mice were taken out. The tissue sections were prepared and stained as the description in Example 3. The stained sections were examined and recorded under a light microscope (Olympus, Tokyo, Japan), and the results were shown in FIG. 14A. The histological images were also further analyzed using the ImageJ software (National Institutes of Health, Bethesda, USA) to determine the linear intercept (represented as Lm in FIG. 14B) to quantify the severity of abnormal lung histopathology and expressed in μm, and the results were shown in FIG. 14B. Data of the experimental results obtained were the mean±IQR of five independent experiments, and the Newman-Keuls multiple comparison post hoc one-way ANOVA analysis was used for statistical analysis; wherein, * represents p value<0.05; **** represents p-value<0.0001, and NS represented not significant.

As shown in FIGS. 14A and 14B, after COPD was induced by cigarette smoke in the mice, compared with the comparison group, the administration of the Hirsutella sinensis polysaccharide of the present invention, the bacterial consortium of the present invention, or the bacterial composition of the present invention containing the Hirsutella sinensis polysaccharide caused the abnormal lung histopathology of the mice significantly relieve to be closer to that of the mice of the control group. The efficacy of the bacterial composition of the present invention containing the Hirsutella sinensis polysaccharide was significantly better than that of the Hirsutella sinensis polysaccharide or the bacterial consortium of the present invention alone. The results indicated that no matter the Hirsutella sinensis polysaccharide of the present invention, the bacterial consortium of the present invention, or the bacterial composition of the present invention containing the Hirsutella sinensis polysaccharide can effectively improve the abnormal lung histopathology in subjects with COPD. In the bacterial composition of the present invention, after the Hirsutella sinensis polysaccharide was utilized and digested by the bacterial consortium, the active substances produced provided significantly improved effects.

5-3 the Hirsutella sinensis Polysaccharide and the Bacterial Composition Containing the Hirsutella sinensis Polysaccharide Improve Abnormal Lung Function Caused by COPD

In one embodiment of the present invention, in order to further observe whether the Hirsutella sinensis polysaccharide of the present invention and the bacterial composition of the present invention containing Hirsutella sinensis polysaccharide can improve the abnormal lung function of subjects with chronic obstructive pulmonary disease, all of the mice of the aforementioned 12-week experiments were evaluated lung functions according to the method described in Example 3.

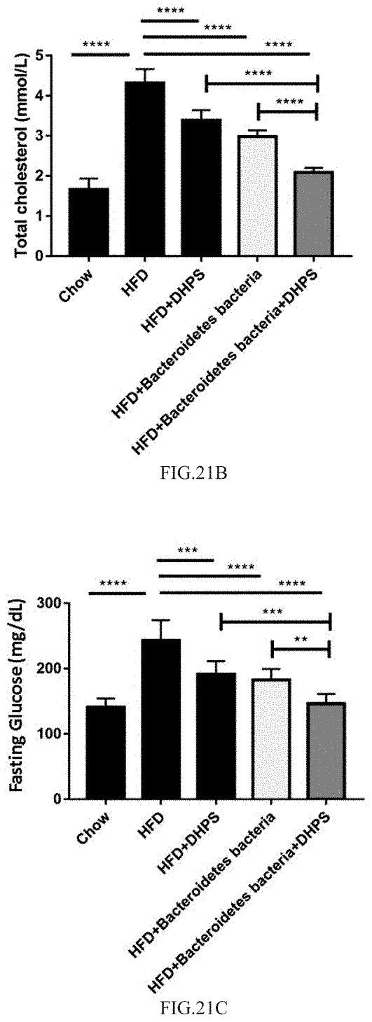

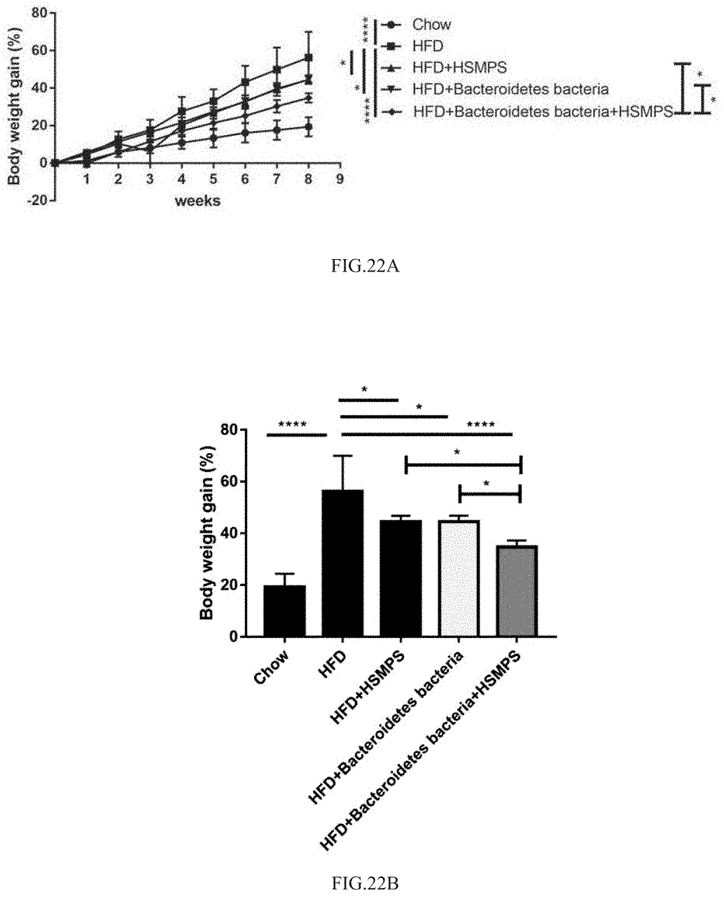

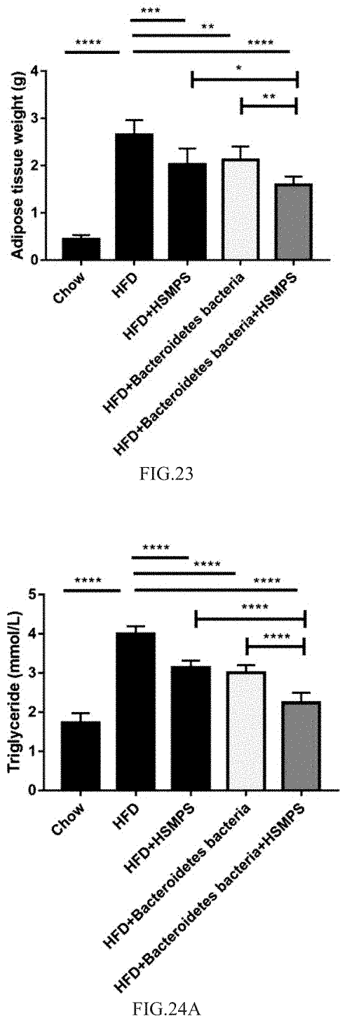

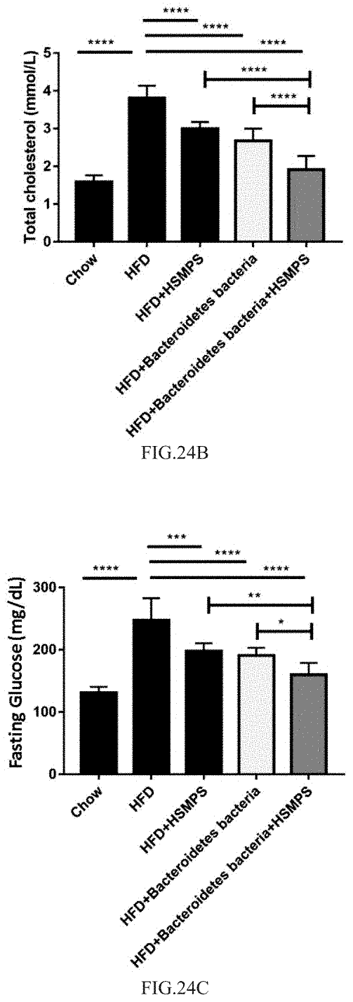

The results of the Hirsutella sinensis polysaccharide of the present invention and the bacterial composition of the present invention containing Hirsutella sinensis polysaccharide on improving abnormality of FRC in the subjects with COPD were shown in FIG. 15A, wherein FRC was expressed in the unit of mL; the results on improving abnormality of FVC in the subjects with COPD were shown in FIG. 15B, wherein FVC was expressed in the unit of mL; the results on improving abnormality of Cchord in the subjects with COPD were shown in FIG. 15C, wherein COPD was expressed in the unit of mL/cm H₂O; and the results on improving abnormality of FEV100/FVC ration in the subjects with COPD were shown in FIG. 15D. All the above maneuvers and perturbations were continuously performed until three correct measurements were achieved. The average of the three measurements of the above parameters for each mouse in each group was used as the result value for that parameter for that group of mice. Data of the experimental results obtained were the mean±IQR of five independent experiments, and the Newman-Keuls multiple comparison post hoc one-way ANOVA analysis was used for statistical analysis; wherein, * represents p value<0.05; ** represents p-value<0.01; *** represents p-value<0.001; **** represents p-value<0.0001, and NS represented not significant.

As shown in FIGS. 15A to 15D, after COPD was induced by cigarette smoke in the mice, compared with the comparison group, the administration of the Hirsutella sinensis polysaccharide of the present invention, the bacterial consortium of the present invention, or the bacterial composition of the present invention containing the Hirsutella sinensis polysaccharide caused FRC, FVC, and Cchord significantly reduce and FEV100/FVC ration significantly increase. The efficacy of the bacterial composition of the present invention containing the Hirsutella sinensis polysaccharide was significantly better than that of the Hirsutella sinensis polysaccharide or the bacterial consortium of the present invention alone. The results indicated that no matter the Hirsutella sinensis polysaccharide of the present invention, the bacterial consortium of the present invention, or the bacterial composition of the present invention containing the Hirsutella sinensis polysaccharide can effectively improve the emphysema in subjects with COPD, and effectively improve the lung function of the subjects with COPD. In the bacterial composition of the present invention, after the Hirsutella sinensis polysaccharide was utilized and digested by the bacterial consortium, the active substances produced provided significantly improved effects.

### Example 6

The Ganoderma lucidum Polysaccharide and the Bacterial Composition Containing the Ganoderma lucidum Polysaccharide Improve Obesity

In one embodiment of the present invention, in order to test the effects of the Ganoderma lucidum polysaccharide of the present invention and the bacterial composition of the present invention containing the Ganoderma lucidum polysaccharide on improving obesity symptoms in a subject, the obese mice induced by being fed with high-fat diets were used as animal model for experiments.

In the embodiment of the present invention, animal experiments were approved by the Institutional Animal Care and Use Committee of Chang Gung University, and the experiments were performed in accordance with the guidelines. The experimental animals used herein were 6 week-old C57BL/6J male mice which were purchased from NLAC (Taipei, Taiwan) and were housed with free access to food and sterile drinking water in a temperature-controlled room (21±2° C.) under a 12-hour dark/light cycle, and were with one-week acclimatization period under this condition.

In the embodiment of the present invention, the Ganoderma lucidum polysaccharides, bacterial consortium, and the bacterial compositions containing the Ganoderma lucidum polysaccharides used in the animal experiments were prepared as described in Example 3. All of the three were incubated in an anaerobic environment at 37° C. for 6 hours before being used to the experimental animals.

After the acclimatization period of the experimental mice was over, the 6-week-old C57BL/6J male mice were separated into the following five groups (n=8): (1) the control group (Chow): the mice were fed with standard chow diet (13.5% of energy from fat; LabDiet, USA, LabDiet 5001), and were administered with 200 μL of PBS orally once a day five times a week for eight weeks; (2) the comparative group (high-fat diet, HFD): the mice were fed with high-fat diet (HFD, 60% of energy from fat; TestDiet, USA, TestDiet 58Y1), and were administered with 200 μL of PBS orally once a day five times a week for eight weeks; (3) the experimental group (HFD+CLPS): the mice were fed with HFD, and were administered with 200 μL of the above-mentioned Ganoderma lucidum polysaccharide of the present invention (25 mg/kg, about 0.5 mg per mouse) orally once a day five times a week for eight weeks; (4) the experimental group (HFD+Bacteroidetes bacteria): the mice were fed with HFD, and were administered with 200 μL of the above-mentioned bacterial consortium of the present invention (3×10¹⁰ CFU/kg, about 6×10⁸ CFU per mouse) orally once a day five times a week for eight weeks; and (5) the experimental group (HFD+Bacteroidetes bacteria+CLPS): the mice were fed with HFD, and were administered with 200 μL of the bacterial composition of the present invention containing the Ganoderma lucidum polysaccharide (25 mg/kg of the Ganoderma lucidum polysaccharide, about 0.5 mg per mouse; 3×10¹⁰ CFU/kg of bacterial consortium, about 6×10⁸ CFU per mouse) orally once a day five times a week for eight weeks.

6-1 the Ganoderma lucidum Polysaccharide and the Bacterial Composition Containing the Ganoderma lucidum Polysaccharide Reduce Body Weight Gain in Obese Subjects

During the 8-week experimental duration, the percentage of body weight gain of each group of mice relative to the starting body weight in the 0th week was monitored every week, and the results were shown in FIG. 16A. Further, in the 8th week of the experiment, the percentage of body weight gain of each group of mice relative to the starting body weight in the 0th week was measured, and the results were shown in FIG. 16B. Data of the experimental results obtained were the mean±SD of eight independent experiments, and the Turkey's multiple comparison post hoc one-way ANOVA analysis was used for statistical analysis; wherein, ** represents p-value<0.01; and **** represents p-value<0.0001.

As shown in FIGS. 16A and 16B, compared with the mice of the control group fed with standard chow diet, the percentage of body weight gain of the mice of the comparison group, in which obesity was induced by being fed with HFD, would significantly increase. However, when obesity was induced in the mice by being fed with HFD, compared with the comparison group, the administration of the Ganoderma lucidum polysaccharide of the present invention, the bacterial consortium of the present invention, or the bacterial composition of the present invention containing the Ganoderma lucidum polysaccharide caused the percentage of body weight gain of the mice significantly reduce. The efficacy of the bacterial composition of the present invention containing the Ganoderma lucidum polysaccharide was significantly better than that of the Ganoderma lucidum polysaccharide or the bacterial consortium of the present invention alone. The results indicated that no matter the Ganoderma lucidum polysaccharide of the present invention, the bacterial consortium of the present invention, or the bacterial composition of the present invention containing the Ganoderma lucidum polysaccharide can effectively reduce body weight gain in obese subjects. In the bacterial composition of the present invention, after the Ganoderma lucidum polysaccharide was utilized and digested by the bacterial consortium, the active substances produced provided significantly improved effects.

6-2 the Ganoderma lucidum Polysaccharide and the Bacterial Composition Containing the Ganoderma lucidum Polysaccharide Reduce Adipose Tissue Weight in Obese Subjects

In the embodiment of the present invention, in order to further observe whether the Ganoderma lucidum polysaccharide of the present invention and the bacterial composition of the present invention containing the Ganoderma lucidum polysaccharide can reduce adipose tissue weight in obese subjects to effectively improve obesity symptoms, the mice of the aforementioned 8-week experiments were sacrificed, and the visceral adipose tissues of each group of mice were taken out and weighed. The results were shown in FIG. 17. Data of the experimental results obtained were the mean±SD of eight independent experiments, and the Turkey's multiple comparison post hoc one-way ANOVA analysis was used for statistical analysis; wherein, ** represents p-value<0.01; and **** represents p-value<0.0001.

As shown in FIG. 17, compared with the mice of the control group fed with standard chow diet, the visceral adipose tissues weight of the mice of the comparison group, in which obesity was induced by being fed with HFD, would significantly increase. However, when obesity was induced in the mice by being fed with HFD, compared with the comparison group, the administration of the Ganoderma lucidum polysaccharide of the present invention, the bacterial consortium of the present invention, or the bacterial composition of the present invention containing the Ganoderma lucidum polysaccharide caused the visceral adipose tissues weight of the mice significantly reduce. The efficacy of the bacterial composition of the present invention containing the Ganoderma lucidum polysaccharide was significantly better than that of the Ganoderma lucidum polysaccharide or the bacterial consortium of the present invention alone. The results indicated that no matter the Ganoderma lucidum polysaccharide of the present invention, the bacterial consortium of the present invention, or the bacterial composition of the present invention containing the Ganoderma lucidum polysaccharide can effectively reduce adipose tissue weight in obese subjects to effectively improve obesity symptoms. In the bacterial composition of the present invention, after the Ganoderma lucidum polysaccharide was utilized and digested by the bacterial consortium, the active substances produced provided significantly improved effects.

6-3 the Ganoderma lucidum Polysaccharide and the Bacterial Composition Containing the Ganoderma lucidum Polysaccharide Reduce Relative Indicators in Obese Subjects

In the embodiment of the present invention, in order to further observe whether the Ganoderma lucidum polysaccharide of the present invention and the bacterial composition of the present invention containing the Ganoderma lucidum polysaccharide can reduce levels of triglyceride, total cholesterol, and fasting glucose in obese subjects to effectively improve obesity symptoms, the mice of the aforementioned 8-week experiments were fasted at the indicated time, and then were anaesthetized and whole blood of each mouse was withdrawn by cardiac puncture. The triglyceride, total cholesterol, and fasting glucose levels were measured by FUJIFILM DRI-CHEM NX-500. The results were shown in FIGS. 18A, 18B, and 18C, respectively. Data of the experimental results obtained were the mean±SD of eight independent experiments, and the Turkey's multiple comparison post hoc one-way ANOVA analysis was used for statistical analysis; wherein, * represents p-value<0.05; ** represents p-value<0.01; and **** represents p-value<0.0001.

As shown in FIGS. 18A to 18C, compared with the mice of the control group fed with standard chow diet, the triglyceride, total cholesterol, and fasting glucose levels in the blood of the mice of the comparison group, in which obesity was induced by being fed with HFD, would significantly increase. However, when obesity was induced in the mice by being fed with HFD, compared with the comparison group, the administration of the Ganoderma lucidum polysaccharide of the present invention, the bacterial consortium of the present invention, or the bacterial composition of the present invention containing the Ganoderma lucidum polysaccharide caused the triglyceride, total cholesterol, and fasting glucose levels in the blood of the mice significantly reduce. The efficacy of the bacterial composition of the present invention containing the Ganoderma lucidum polysaccharide was significantly better than that of the Ganoderma lucidum polysaccharide or the bacterial consortium of the present invention alone. The results indicated that no matter the Ganoderma lucidum polysaccharide of the present invention, the bacterial consortium of the present invention, or the bacterial composition of the present invention containing the Ganoderma lucidum polysaccharide can effectively reduce the levels of triglyceride, total cholesterol, and fasting glucose in obese subjects to effectively improve obesity symptoms. In the bacterial composition of the present invention, after the Ganoderma lucidum polysaccharide was utilized and digested by the bacterial consortium, the active substances produced provided significantly improved effects.

### Example 7

The Dendrobium huoshanense Polysaccharide and the Bacterial Composition Containing the Dendrobium huoshanense Polysaccharide Improve Obesity

In one embodiment of the present invention, in order to test the effects of the Dendrobium huoshanense polysaccharide of the present invention and the bacterial composition of the present invention containing the Dendrobium huoshanense polysaccharide on improving obesity symptoms in a subject, the obese mice induced by being fed with high-fat diets were also used as animal model for experiments.

In the embodiment of the present invention, the experimental animals used herein were also 6-week-old C57BL/6J male mice, and the mice were housed under the conditions described in Example 6.

In the embodiment of the present invention, the Dendrobium huoshanense polysaccharides, bacterial consortium, and the bacterial compositions containing the Dendrobium huoshanense polysaccharides used in the animal experiments were prepared as described in Example 4. All of the three were incubated in an anaerobic environment at 37° C. for 6 hours before being used to the experimental animals.

After the acclimatization period of the experimental mice was over, the 6-week-old C57BL/6J male mice were separated into the following five groups (n=8): (1) the control group (Chow): the mice were fed with standard chow diet, and were administered with 200 μL of PBS orally once a day five times a week for eight weeks; (2) the comparative group (high-fat diet, HFD): the mice were fed with HFD, and were administered with 200 μL of PBS orally once a day five times a week for eight weeks; (3) the experimental group (HFD+DHPS): the mice were fed with HFD, and were administered with 200 μL of the above-mentioned Dendrobium huoshanense polysaccharide of the present invention (25 mg/kg, about 0.5 mg per mouse) orally once a day five times a week for eight weeks; (4) the experimental group (HFD+Bacteroidetes bacteria): the mice were fed with HFD, and were administered with 200 μL of the above-mentioned bacterial consortium of the present invention (3×10¹⁰ CFU/kg, about 6×10⁸ CFU per mouse) orally once a day five times a week for eight weeks; and (5) the experimental group (HFD+Bacteroidetes bacteria+DHPS): the mice were fed with HFD, and were administered with 200 μL of the bacterial composition of the present invention containing the Dendrobium huoshanense polysaccharide (25 mg/kg of the Dendrobium huoshanense polysaccharide, about 0.5 mg per mouse; 3×10¹⁰ CFU/kg of bacterial consortium, about 6×10⁸ CFU per mouse) orally once a day five times a week for eight weeks.

7-1 the Dendrobium huoshanense Polysaccharide and the Bacterial Composition Containing the Dendrobium huoshanense Polysaccharide Reduce Body Weight Gain in Obese Subjects

During the 8-week experimental duration, the percentage of body weight gain of each group of mice relative to the starting body weight in the 0th week was monitored every week, and the results were shown in FIG. 19A. Further, in the 8th week of the experiment, the percentage of body weight gain of each group of mice relative to the starting body weight in the 0th week was measured, and the results were shown in FIG. 19B. Data of the experimental results obtained were the mean±SD of eight independent experiments, and the Turkey's multiple comparison post hoc one-way ANOVA analysis was used for statistical analysis; wherein, * represents p-value<0.05; ** represents p-value<0.01; and **** represents p-value<0.0001.

As shown in FIGS. 19A and 19B, when obesity was induced in the mice by being fed with HFD, compared with the comparison group, the administration of the Dendrobium huoshanense polysaccharide of the present invention, the bacterial consortium of the present invention, or the bacterial composition of the present invention containing the Dendrobium huoshanense polysaccharide caused the percentage of body weight gain of the mice significantly reduce. The efficacy of the bacterial composition of the present invention containing the Dendrobium huoshanense polysaccharide was significantly better than that of the Dendrobium huoshanense polysaccharide or the bacterial consortium of the present invention alone. The results indicated that no matter the Dendrobium huoshanense polysaccharide of the present invention, the bacterial consortium of the present invention, or the bacterial composition of the present invention containing the Dendrobium huoshanense polysaccharide can effectively reduce body weight gain in obese subjects. In the bacterial composition of the present invention, after the Dendrobium huoshanense polysaccharide was utilized and digested by the bacterial consortium, the active substances produced provided significantly improved effects.

7-2 the Dendrobium huoshanense Polysaccharide and the Bacterial Composition Containing the Dendrobium huoshanense Polysaccharide Reduce Adipose Tissue Weight in Obese Subjects

In the embodiment of the present invention, in order to further observe whether the Dendrobium huoshanense polysaccharide of the present invention and the bacterial composition of the present invention containing the Dendrobium huoshanense polysaccharide can reduce adipose tissue weight in obese subjects to effectively improve obesity symptoms, the mice of the aforementioned 8-week experiments were sacrificed, and the visceral adipose tissues of each group of mice were taken out and weighed. The results were shown in FIG. 20. Data of the experimental results obtained were the mean±SD of eight independent experiments, and the Turkey's multiple comparison post hoc one-way ANOVA analysis was used for statistical analysis; wherein, * represents p-value <0.05; ** represents p-value<0.01; *** represents p-value<0.001; and **** represents p-value<0.0001.

As shown in FIG. 20, when obesity was induced in the mice by being fed with HFD, compared with the comparison group, the administration of the Dendrobium huoshanense polysaccharide of the present invention, the bacterial consortium of the present invention, or the bacterial composition of the present invention containing the Dendrobium huoshanense polysaccharide caused the visceral adipose tissues weight of the mice significantly reduce. The efficacy of the bacterial composition of the present invention containing the Dendrobium huoshanense polysaccharide was significantly better than that of the Dendrobium huoshanense polysaccharide or the bacterial consortium of the present invention alone. The results indicated that no matter the Dendrobium huoshanense polysaccharide of the present invention, the bacterial consortium of the present invention, or the bacterial composition of the present invention containing the Dendrobium huoshanense polysaccharide can effectively reduce adipose tissue weight in obese subjects to effectively improve obesity symptoms. In the bacterial composition of the present invention, after the Dendrobium huoshanense polysaccharide was utilized and digested by the bacterial consortium, the active substances produced provided significantly improved effects.

7-3 the Dendrobium huoshanense Polysaccharide and the Bacterial Composition Containing the Dendrobium huoshanense Polysaccharide Reduce Relative Indicators in Obese Subjects

In the embodiment of the present invention, in order to further observe whether the Dendrobium huoshanense polysaccharide of the present invention and the bacterial composition of the present invention containing the Dendrobium huoshanense polysaccharide can reduce levels of triglyceride, total cholesterol, and fasting glucose in obese subjects to effectively improve obesity symptoms, after the aforementioned 8-week experiments, whole blood of each mouse was withdrawn as described in Example 6. The triglyceride, total cholesterol, and fasting glucose levels were measured by FUJIFILM DRI-CHEM NX-500. The results were shown in FIGS. 21A, 21B, and 21C, respectively. Data of the experimental results obtained were the mean±SD of eight independent experiments, and the Turkey's multiple comparison post hoc one-way ANOVA analysis was used for statistical analysis; wherein, * represents p-value<0.05; ** represents p-value<0.01; *** represents p-value<0.001; and **** represents p-value<0.0001.

As shown in FIGS. 21A to 21C, when obesity was induced in the mice by being fed with HFD, compared with the comparison group, the administration of the Dendrobium huoshanense polysaccharide of the present invention, the bacterial consortium of the present invention, or the bacterial composition of the present invention containing the Dendrobium huoshanense polysaccharide caused the triglyceride, total cholesterol, and fasting glucose levels in the blood of the mice significantly reduce. The efficacy of the bacterial composition of the present invention containing the Dendrobium huoshanense polysaccharide was significantly better than that of the Dendrobium huoshanense polysaccharide or the bacterial consortium of the present invention alone. The results indicated that no matter the Dendrobium huoshanense polysaccharide of the present invention, the bacterial consortium of the present invention, or the bacterial composition of the present invention containing the Dendrobium huoshanense polysaccharide can effectively reduce the levels of triglyceride, total cholesterol, and fasting glucose in obese subjects to effectively improve obesity symptoms. In the bacterial composition of the present invention, after the Dendrobium huoshanense polysaccharide was utilized and digested by the bacterial consortium, the active substances produced provided significantly improved effects.

### Example 8

The Hirsutella sinensis Polysaccharide and the Bacterial Composition Containing the Hirsutella sinensis Polysaccharide Improve Obesity

In one embodiment of the present invention, in order to test the effects of the Hirsutella sinensis polysaccharide of the present invention and the bacterial composition of the present invention containing the Hirsutella sinensis polysaccharide on improving obesity symptoms in a subject, the obese mice induced by being fed with high-fat diets were also used as animal model for experiments.

In the embodiment of the present invention, the experimental animals used herein were also 6-week-old C57BL/6J male mice, and the mice were housed under the conditions described in Example 6.

In the embodiment of the present invention, the Hirsutella sinensis polysaccharides, bacterial consortium, and the bacterial compositions containing the Hirsutella sinensis polysaccharides used in the animal experiments were prepared as described in Example 5. All of the three were incubated in an anaerobic environment at 37° C. for 6 hours before being used to the experimental animals.

After the acclimatization period of the experimental mice was over, the 6-week-old C57BL/6J male mice were separated into the following five groups (n=8): (1) the control group (Chow): the mice were fed with standard chow diet, and were administered with 200 μL of PBS orally once a day five times a week for eight weeks; (2) the comparative group (high-fat diet, HFD): the mice were fed with HFD, and were administered with 200 μL of PBS orally once a day five times a week for eight weeks; (3) the experimental group (HFD+HSMPS): the mice were fed with HFD, and were administered with 200 μL of the above-mentioned Hirsutella sinensis polysaccharide of the present invention (25 mg/kg, about 0.5 mg per mouse) orally once a day five times a week for eight weeks; (4) the experimental group (HFD+Bacteroidetes bacteria): the mice were fed with HFD, and were administered with 200 μL of the above-mentioned bacterial consortium of the present invention (3×10¹⁰ CFU/kg, about 6×10⁸ CFU per mouse) orally once a day five times a week for eight weeks; and (5) the experimental group (HFD+Bacteroidetes bacteria+HSMPS): the mice were fed with HFD, and were administered with 200 μL of the bacterial composition of the present invention containing the Hirsutella sinensis polysaccharide (25 mg/kg of the Hirsutella sinensis, about 0.5 mg per mouse; 3×10¹⁰ CFU/kg of bacterial consortium, about 6×10⁸ CFU per mouse) orally once a day five times a week for eight weeks.

8-1 the Hirsutella sinensis Polysaccharide and the Bacterial Composition Containing the Hirsutella sinensis Polysaccharide Reduce Body Weight Gain in Obese Subjects

During the 8-week experimental duration, the percentage of body weight gain of each group of mice relative to the starting body weight in the 0th week was monitored every week, and the results were shown in FIG. 22A. Further, in the 8th week of the experiment, the percentage of body weight gain of each group of mice relative to the starting body weight in the 0th week was measured, and the results were shown in FIG. 22B. Data of the experimental results obtained were the mean±SD of eight independent experiments, and the Turkey's multiple comparison post hoc one-way ANOVA analysis was used for statistical analysis; wherein, * represents p-value<0.05; and **** represents p-value<0.0001.

As shown in FIGS. 22A and 22B, when obesity was induced in the mice by being fed with HFD, compared with the comparison group, the administration of the Hirsutella sinensis polysaccharide of the present invention, the bacterial consortium of the present invention, or the bacterial composition of the present invention containing the Hirsutella sinensis polysaccharide caused the percentage of body weight gain of the mice significantly reduce. The efficacy of the bacterial composition of the present invention containing the Hirsutella sinensis polysaccharide was significantly better than that of the Hirsutella sinensis polysaccharide or the bacterial consortium of the present invention alone. The results indicated that no matter the Hirsutella sinensis polysaccharide of the present invention, the bacterial consortium of the present invention, or the bacterial composition of the present invention containing the Hirsutella sinensis polysaccharide can effectively reduce body weight gain in obese subjects. In the bacterial composition of the present invention, after the Hirsutella sinensis polysaccharide was utilized and digested by the bacterial consortium, the active substances produced provided significantly improved effects.

8-2 the Hirsutella sinensis Polysaccharide and the Bacterial Composition Containing the Hirsutella sinensis Polysaccharide Reduce Adipose Tissue Weight in Obese Subjects

In the embodiment of the present invention, in order to further observe whether the Hirsutella sinensis polysaccharide of the present invention and the bacterial composition of the present invention containing the Hirsutella sinensis polysaccharide can reduce adipose tissue weight in obese subjects to effectively improve obesity symptoms, the mice of the aforementioned 8-week experiments were sacrificed, and the visceral adipose tissues of each group of mice were taken out and weighed. The results were shown in FIG. 23. Data of the experimental results obtained were the mean±SD of eight independent experiments, and the Turkey's multiple comparison post hoc one-way ANOVA analysis was used for statistical analysis; wherein, * represents p-value<0.05; ** represents p-value<0.01; *** represents p-value<0.001; and **** represents p-value<0.0001.

As shown in FIG. 23, when obesity was induced in the mice by being fed with HFD, compared with the comparison group, the administration of the Hirsutella sinensis polysaccharide of the present invention, the bacterial consortium of the present invention, or the bacterial composition of the present invention containing the Hirsutella sinensis polysaccharide caused the visceral adipose tissues weight of the mice significantly reduce. The efficacy of the bacterial composition of the present invention containing the Hirsutella sinensis polysaccharide was significantly better than that of the Hirsutella sinensis polysaccharide or the bacterial consortium of the present invention alone. The results indicated that no matter the Hirsutella sinensis polysaccharide of the present invention, the bacterial consortium of the present invention, or the bacterial composition of the present invention containing the Hirsutella sinensis polysaccharide can effectively reduce adipose tissue weight in obese subjects to effectively improve obesity symptoms. In the bacterial composition of the present invention, after the Hirsutella sinensis polysaccharide was utilized and digested by the bacterial consortium, the active substances produced provided significantly improved effects.

8-3 the Hirsutella sinensis Polysaccharide and the Bacterial Composition Containing the Hirsutella sinensis Polysaccharide Reduce Relative Indicators in Obese Subjects

In the embodiment of the present invention, in order to further observe whether the Hirsutella sinensis polysaccharide of the present invention and the bacterial composition of the present invention containing the Hirsutella sinensis polysaccharide can reduce levels of triglyceride, total cholesterol, and fasting glucose in obese subjects to effectively improve obesity symptoms, after the aforementioned 8-week experiments, whole blood of each mouse was withdrawn as described in Example 6. The triglyceride, total cholesterol, and fasting glucose levels were measured by FUJIFILM DRI-CHEM NX-500. The results were shown in FIGS. 24A, 24B, and 24C, respectively. Data of the experimental results obtained were the mean±SD of eight independent experiments, and the Turkey's multiple comparison post hoc one-way ANOVA analysis was used for statistical analysis; wherein, * represents p-value<0.05; ** represents p-value<0.01; *** represents p-value<0.001; and **** represents p-value<0.0001.

As shown in FIGS. 24A to 24C, when obesity was induced in the mice by being fed with HFD, compared with the comparison group, the administration of the Hirsutella sinensis polysaccharide of the present invention, the bacterial consortium of the present invention, or the bacterial composition of the present invention containing the Hirsutella sinensis polysaccharide caused the triglyceride, total cholesterol, and fasting glucose levels in the blood of the mice significantly reduce. The efficacy of the bacterial composition of the present invention containing the Hirsutella sinensis polysaccharide was significantly better than that of the Hirsutella sinensis polysaccharide or the bacterial consortium of the present invention alone. The results indicated that no matter the Hirsutella sinensis polysaccharide of the present invention, the bacterial consortium of the present invention, or the bacterial composition of the present invention containing the Hirsutella sinensis polysaccharide can effectively reduce the levels of triglyceride, total cholesterol, and fasting glucose in obese subjects to effectively improve obesity symptoms. In the bacterial composition of the present invention, after the Hirsutella sinensis polysaccharide was utilized and digested by the bacterial consortium, the active substances produced provided significantly improved effects.

In summary, the present invention proved that the bacterial consortium consisted of Bacteroidetes can effectively utilize and digest the traditional Chinese medicine polysaccharide of the present invention, and proved that the traditional Chinese medicine polysaccharide of the present invention and the bacterial composition of the present invention containing the traditional Chinese medicine polysaccharide can effectively inhibit inflammatory responses, improve chronic obstructive pulmonary disease, and prevent and/or treat diet-induced obesity and metabolic syndrome. In addition, previous studies have shown that synbiotic which was prepared by mixing polysaccharides and bacteria could not maintain the effects of the polysaccharides or the bacteria themselves. However, the results of the present invention show that the bacterial composition of the present invention which was prepared by mixing the traditional Chinese medicine polysaccharides and the bacterial consortium of the present invention would provide the significantly better effects.

What is claimed is:

1. A method of ameliorating chronic obstructive pulmonary disease, comprising administering to a subject in need thereof a composition containing a traditional Chinese medicine polysaccharide, wherein the traditional Chinese medicine polysaccharide is selected from a group consisting of Ganoderma lucidum polysaccharide, Dendrobium huoshanense polysaccharide, Hirsutella sinensis polysaccharide, and any combination thereof.

2. The method according to claim 1, wherein the Ganoderma lucidum polysaccharide is from a water extract of Ganoderma lucidum, the Dendrobium huoshanense polysaccharide is from a water extract of Dendrobium huoshanense, and the Hirsutella sinensis polysaccharide is from a water extract of Hirsutella sinensis.

3. The method according to claim 1, wherein an effective amount of the traditional Chinese medicine polysaccharide is at least twice a week for the subject in need thereof with at least 25 mg/kg each time.

4. The method according to claim 1, wherein the composition is ameliorating body weight loss, abnormal lung histopathology, and/or abnormal lung function of the subject caused by chronic obstructive pulmonary disease.

5. The method according to claim 1, wherein the composition further comprises a pharmaceutically acceptable excipient, carrier, adjuvant, or food additive.

6. The method according to claim 1, wherein the composition is in the form of a spray, a solution, a semi-solid preparation, a solid preparation, a gelatin capsule, a soft capsule, a tablet, a chewing gum, or a freeze-dried powder preparation.

7. A method of ameliorating chronic obstructive pulmonary disease, comprising administering to a subject in need thereof a bacterial composition containing a bacterial consortium and the traditional Chinese medicine polysaccharide according to claim 1.

8. The method according to claim 7, wherein the bacterial consortium is consisted of a Bacteroidetes bacterium.

9. The method according to claim 8, wherein the Bacteroidetes bacterium is selected from a group consisting of Parabacteroides goldsteinii (P. goldsteinii), Parabacteroides distasonis (P. distasonis), Bacteroides xylanisolvens (B. xylanisolvens), Bacteroides uniformis (B. uniformis), Bacteroides fragilis (B. fragilis), Bacteroides ovatus (B. ovatus), and any combination thereof.

10. The method according to claim 7, wherein an effective amount of the bacterial composition is at least twice a week for the subject in need thereof with the bacterial composition containing at least 3×10¹⁰ CFU/kg of the bacterial consortium and at least 25 mg/kg of the bacterial composition each time.

11. The method according to claim 7, wherein the bacterial composition is ameliorating body weight loss, abnormal lung histopathology, and/or abnormal lung function of the subject caused by chronic obstructive pulmonary disease.

12. The method according to claim 7, wherein the bacterial composition further comprises a pharmaceutically acceptable excipient, carrier, adjuvant, or food additive.

13. The method according to claim 7, wherein the bacterial composition is in the form of a spray, a solution, a semi-solid preparation, a solid preparation, a gelatin capsule, a soft capsule, a tablet, a chewing gum, or a freeze-dried powder preparation.

14. A method of preventing and/or treating obesity, comprising administering to a subject in need thereof a bacterial composition containing a bacterial consortium and the traditional Chinese medicine polysaccharide according to claim 1.

15. The method according to claim 14, wherein the bacterial consortium is consisted of a Bacteroidetes bacterium.

16. The method according to claim 15, wherein the Bacteroidetes bacterium is selected from a group consisting of P. goldsteinii, P distasonis, B. xylanisolvens, B. uniformis, B. fragilis, B. ovatus, and any combination thereof.

17. The method according to claim 14, wherein the bacterial composition reduces body weight gain of the subject in need thereof.

18. The method according to claim 14, wherein the bacterial composition reduces adipose tissue weight, triglyceride, total cholesterol, and/or fasting glucose of the subject in need thereof.

19. The method according to claim 14, wherein an effective amount of the bacterial composition is at least twice a week for the subject in need thereof with the bacterial composition containing at least 3×10¹⁰ CFU/kg of the bacterial consortium and at least 25 mg/kg of the bacterial composition each time.

20. The method according to claim 14, wherein the bacterial composition further comprises a pharmaceutically acceptable excipient, carrier, adjuvant, or food additive.

21. The method according to claim 14, wherein the bacterial composition is in the form of a spray, a solution, a semi-solid preparation, a solid preparation, a gelatin capsule, a soft capsule, a tablet, a chewing gum, or a freeze-dried powder preparation.
